SEQUENCE LISTING (19 sequences)

SEQ ID NO: 1 - protein, 1335 aa, unidentified
MKSLAQFQNLYALQKTLRFELKPEGHTRETFNRWLEEIEKEQASENENIVYQDLLRAKKYEKIKIILDEYHKDFIEQALAYANLTELEKYEELYRKSNRTSEEEEEFENTKESLRKQIANIFIKNPNKTVQERWKFLFSKKLIQNELIVWVKGNYELLSEKLKNEFPDESSIISTIEDFKYFTTYFRNYHENRKNLYSNEDKFSTIAHRLIHENLPKFIDNIAIYQKAKAVLNINEVEKELGLPEDTLDKIFSLDFFSKALTQKGIDQYNYFLGGKTENEVKKIKGLNEFINLYNQQQQDKNQRLPFLKVLYKLPLFERTSTSFRFEPIENDRDLIERIGKFYYNDLKQYRDDSQGDTTDILSGINTLLRHVHDYRDGLYVNGGITLTQISQKIFGSWSYINNALAYFYDTYIDASGVDHQGERKPKKQKQIQEKTKWLKQKQFPVILVEKALSEYKSIETNEDLKTRISDTTLCDFFKRCGNDDNGQDLFDRIEARLREKNEEGYSLEDLLKKEFTTERKLMQDKTKTLLIKNFLDVIQGDKDDITAGLLHFVKCLIPRTEISEKNELFYSGMEKYLNILSEVTPLYNKARNYLTQKPYSIEKVKLNFENSTLLDGWDENEESDNSCVLLRKRGYYYLGIMNKKHNMIFDRKIYPKATEGEAYYEKMIYKLLPGAYKMLPKVFFSEKNIDYFKPSEEILRIRNTASYSKNGQPQEGYQKASFSIEDCRKYIDFFKKCIANHWDWQKFNFNFSPTEYYQSIDEFYREIERQGYKIDFVKIPESYINQLIKENKLYLFKIYNKDFSEKKKSKGKDNLHTLYWKMLFDEKNLKDVVLKLNGEAEVFFRQKSILYNEEIWNKGHHYSELKDRFSYPIISNKRYAEDKFFLHVPITLNFKADGINNVNNMVNEFIKDNRDIHIIGIDRGERHLLYVSVINQKGDIVEQCSLNEIVTEYNGKIFKKNYHEELDNLEKERDRARKDWQTIANIKELKEGYLSHVIHKISKLILKYNAIVVMEDLNSGFKRGRQKVEKQVYQNFEKQLIEKLNYLVLKESNVDEPGGVLRAYQLANKFETFKKLGKQSGIIFYVPAAYTSAIDPVTGYIQYLYPLKQADSVEKARKFYSQFKRISYNPHKQWFEFSFDYNDFNIIYHGKSSWTICTTNTERYMWNRLLNNGHGGEELVYVTNELELLFGEYNIIYGDGKDIKQQITDVQDIDVDRTAKQFYKRINELLNLTLKLRHNNGKKGADEEDYILSPVEPYFDSRFESRKPSMQQTLPINADANGAFNIARKGLLLLERLNQLGVEEFEKTKKSNNKKTQWLPHELWVEYAQNHTRK

SEQ ID NO: 2 - protein, 1273 aa, unidentified
MQDKTGWSSFTNKYSLSKTLRFELKPVGNTQKMLEDDGVFQKDRERQENYKKVKPFMDKLHREFIKEALNNLKLEGLTEYFEIFKKFRKDKNNKELKNAEKKLRQIIGRCYTETAQIWVEKYKEFGFKKKNIGFLFEEGVFELMKLKYGNDEASQIEKNGEVLSIFDGWKGFLGYFKKFFETRNNFYKDDGTSTAVSTRIINENLKIYLDNLIKYNKIKDKVDFKEADILQENKLNLSDFFNVESYAKYSLQKGIDYYNEILGGKTLKNGTKLKGLNEVINEYKQKNKSGELSKFKMLKKQILGEGEDRTLFEEIENEDELKDVLKDFFYNADPKITLFKTLLEDFFSNTEKYKDELDKIYFNTVAINGILHRWVDDSGVFQKYLFEVLKSNKLVKSNHYDKKEDSYKFPDFISFEHIKVALENCERDGLKDKFWKEKYYTKECLTENGLANLWQEFLEIYKCEFKKLYDYKTDDNDCYLQYRDNYKKYILDANFNPKEKSAKDIIKDYLDSVLSIYQLAKYFALEKKKVWTTDYETGDFYYEYIKFYEDTYEQIIKPYNLVRNYLTRKPINTAKKWKLNFDNAYLASGWDKDKEVSNLTVILRRDEQYYLAIMKKGKNKIFEKKFSCGEFEKMEYKQIAEASSDIHNLVLMNDGSCRRCIKMHDKRKYWPLDISIIKEKKSYAKENFVRRDFERFVNYMKKCSLLYWKEYDLKFSDTSTYKNINDFTNEIASQGYKLSFSAIPESYINEKNNNGELYLFQIYNKDFGIKTEGNKNLHTMYWESIFSEENRFRNFIVKLNGKAEIFYRPKSEQVEKEQRNFTREIIKNRRYTENKIYFHCPITLNRISRENVKKFNNGINNYIATNPNINILGVDRGEKHLVYYAIVDQDGKLIDAEDATGSFNTIGSTDYHRLLEEKAKDREKERKDWDLIRGIKDLKKGYISLVVRKIADLAIKYNAIIIFEDLNTRFKQIRGGMEKSVYQQLEKALINKLSFLVNKGEKDPEQAGHLLKAYQLAAPFQTFDKMGRQTGIIFYTQASYTSKIDPITGWRPNLYLKYRNIDDSKESIKKFKSILFNKEKNRFEFTYDLKDFVDFEEDKIPEKTEWTLCSSVERHKWNRHMNNNKGGYEVYKDLTENFYKLFDENNISMNKDIVDQVESISNGNFFRQFIYLFNLVCQIRNTDEKAEDVDKRDFILSPVEPFFDSRRAKDFKAYGDNLPKNGDENGAYNIARKGVLIIKKIKEYYNQNGSCDKLGWGDLSISHKEWDDFATNN

SEQ ID NO: 3 - protein, 1301 aa, unidentified
MDSYEQFTKLYPIQKTIRFELKPQGRTKEHFDNSNFLEKDRERDDNYKILKEVIDDYHREFIDECLSNIQLNWDDLKKFSEEYRRSKEKKNNRDSESEQKRMSTTSETRAINKKNLEAEQKRMRGEIVSAFKKDDRFKHLFSEKLFSILLKNQIYEKGTLEEIEAFDCFNKFSGYFKSFHENRKNMYSDEDKETAISYRIINENFPKLLDNFEKYQYVCREYPEQIREAESTLAEAGCYIKMDEIFSIDNFNNVMMQGGKESGISRYNLAIGGIVQGTGEKPKGLNEFLNLAYQNEPNGRKKIRMEPLYKQILSKEESFSYRLEAFTDDSQLLSAIRSFFDIVEKDKNGNIFDRAVNLMSSFSNYDTSKIYIRKAYLNQVSKEIFGYRGKSDSKPAKTADESLNKSGGWEKLGQMLRDYKADSIGDRNLEKTCKKVDKWLDSDEFTLSDILGAISLAGSNETFEAYVSEICVARRNIDKEKEKEKNINVEKISGDTESIQIIKALLDSVQEFFHLLSPFQLHPNTPHDWTFYAEFNDIYDKLSAITPLYNQARNHLTKKNLDTSKIKLNFNNPTLANGWDVNKEYENTAVILIRDGKYYLGIMNPKNKRKIKFDEGSGAGPFYQKMVYKLLPGPYRMLPKVFFAKKNIDYYNPSQEIREGYKAGKHKKGKEFDKGFCHKLIDFFKESIQKNENWKVFDFKFSPTESYDDISEFYQEVEKQGYRMYFVNIPSDTIDRYVEGGDMFLFQIYNKDFAKGAKGNKDMHTLYWNAVFSEENLQKGVMKLSGEAELFYRKKSDIKDPPHREGEILVNRTYIDRTHVSGVMGEQNTVKESRIPVPDEIHKNLFDYYNHGRELTKEEKEYCDKVGSFKAYYGIVKDRRYLENKMYFHVPLTLNFKAIGEKRINKMAIEKFLTDENACIIGIDRGERNLLYYSIIDRNGKIIDQKSLNVIDGFDYHEKLSQRQTEREVARQSWNSIGKIKDLKEGYLAKAVHEISKMAIKYNAIVVLEDLHFGFKKGRLKVEKQIYQKFEEMLINKLNYLVFKDVSDSSDAGGVLNAYQLTAPLESFSKLGKQSGILFYVPAAFTSVIDPTTGFVDLFNSSSITSTQKKKEFLQRFESIVYSARDGGIFAFTFDYRNFSKIATDHRNMWTVYTHGERIRYVRDEKCYKTTDPTKRIKEALSGIEYDDGSDIRDKITQSGDNNLINTVYHSFMDTIKMRNKDGRIDYIISPVKNRNGEFFRSDYKHRDFPVDADANGAYHIALKGELLMRMIGKTYDSNSDKMPKLEHKDWFEFMQTRGDQ

SEQ ID NO: 4 - protein, 1188 aa, unidentified
MCVSRLPWFNITLTGKLNRQRLNQMCVSRLPWFCTPKGQLAATPKTVVAQQENAMLAIIRDVHEAAPADLKTVAQRLEPGYFVTQFPKQQMTGDEARAEAERLFAACQKKFKELAEYEDGYRQCLDALGPNLSLPRLGRKPKGAYPYAVVFKLMPTNATWECFKRVTASLYKRAQKGVVSPVSADSIADVRTNDEPLFEYFTNLALVRPPGNKDRAVWFEFDLAAFIEAIKSPHQFFQDTIKREQAVAQIKAKLDAMDGQGRAASGEEDALPGFEGDDRITLLRELVTDTLGYLAEADASTSPGGKIEYSIQERTVRGFAEVKRRWRDLVEKGKATEDALLKVLAEEQTEHRDDFGSATLYRELAKPKFQPIWRDPGTQPWHADDPLRAWLEYRELGRELEDKQRPIRFTPVHPVHSPRFFIFPKKKGGGRFGTVHEPGQLRVMAGIVAQTQHGWEPVPVRITYAAPRLRRDQLRDDVETDLESRPWLQPMMQALGLPEPDTADFSNCRVTLQPSAPDDIQLTFPVDVSADKLTTAIGKAARWAKQFNLFPDGDNFYNASLRWPHEKKPSKPPVPWHEALDNFSVLAADLGQRCAGAFARLEVRANDDFAGKPSRFIGETPGKKWRAALVAAGMLRLPGEEQTVWRPGATGPNFHTELSGSRGRMARPHEADDTADLLRAFDCPEESLMPADWRTSLSFPEQNDKLLVAARRYQSRLARLHRWCWFLTDEKKRQTALDEIREAEDMPAADDPQLTDKLRALLLQKQAALPGLLVRLANRILPLRGRSWQWETHPDKADCHLLTQTGPALPDVWIRGQRGLSMQRIEQIEELRRRFQSLNQMQRREIGGKPPIRRDDSIPDCCPDLLDKLDQIKEQRANQAAHMILAEALGLRLAPPPADKRQLRASRDVHGQYVKSREPVDFIVIEDLSRYRSSQGRAPRENSRLMKWCHRAVRDKLRELCEPFGIPVVETPAAYSSRFCSRSGVAGFRAVEVGPGFDREFPWMMLKDREDEGEPVRQLILQVATLNQGRDGKPPRTLLAPLAGGPIFVPIVDKLNGADIQPALAQADINAAINLGLRAIADPRLWSIHPRCRTQRQGDQMLTREKRKFGETGQPLAVHRADGVKPDDTRNPNFFADISGSLPAWESATLDGQHLLSGRCLRSEIKKRQWQRCAEINDRRMNRWMKGE

SEQ ID NO: 5 - protein, 1518 aa, unidentified
MTELQTQRAYTLRLKGIDEKDQSWRDALWKTHEAVNKGAKVFGDWLLTLRGGLDHTLADAEIPGEKGKPDRAPTQEERKHRRILLALSWLSVESERGAPEEFIVATGKEPAATRNDKVIAALKDILRGRNLTEEKISEWTEVCTPSLSAAIREDAVWVNRSRAFDEAVKRIGSSLTREEVWDMLECFFGSRNAYLAPVKISEDESSDGEQEEKAKDLVQKAGQWLSSRFGTGEGADFAKMAAVYAKIAAWAGNAQAGTTGNEVINNLATALREFTPKSNDLKGVLDLISGPGYKSATRNLLKQIANTKTVTREDISKLQETAGEDSEECATKTGSKGKRAYADAILKDVESVCGFTYRIDKDGQPVSVADYSKYDEDYKWGSSRHKEFAVMLDHAARRVSLAHTWIKRAEAERRKFEEDSKKIMQVPQAAKDWLDAYCAQRSEASGALEPYRIRKRAIQGWKEIIASWNKPDCKTAEDRIAAARQLQDDPEIEKFGDIQLFEALAEDDAQCVWKKEDGTLDPEILINYTLASEAMFKKQHFKVPSYRHPDAFLYPVFCDFGNSRWELDFSIREAATKLKEIEAKIEKQRQEVHKVQQALEKCENDEKRPKMEERLKEAQKKLQESQNYGEYLHSNNKITMVLFDGTFVKKHIFAWQSKRLTKDLALYQEPSADPKNVVSRADRLGRAVASVGINDAVKVAGLFEQENWNGRLQAPRQQLEAIAQYVEKHGWDNKAEKMRASIKWFITFSAKLQSKGPWNEFARKHGLKEDPHYWPHAEKNENRTAHSRLILSRLPGLRVLSVDLGHRYAAACAVWEALGSEAFKKDIEGKRIIRGDTDENALYCHTEHEANGKKHITIYRRIGADTLPDGAHHPAPWARLDRQFLIKLQGEDEQAREASNEEIWKVHQLENTLGRRTPLIDRLIAGGWGYTEKQKARLEVLTNLGWCPTNKTDNQEEGDEEETAILSKPSLLVDDLMFSAVRTLRLALKRHGDRARIAHYLITDEKTKPGGVKEKLDKNGRVELLLDALGLWHDLFSSPGWHDEKAKQLWNAYIAGLLPEGELQQAKSVTTSAALGGQQKKEKKEKLRAVAEALYLNSDLCHSLNEVWRKRWEEDDKQWRIYIRWFKDWIMPRGANAKSPAIRHVGGLSLTRLATLTEFRRKVQVGFFTRLHPDGTKTETREDFGQKTLDTLEHLREQRVKQLASRIVEAALGIGSEDKRHWDGKKRPRQRIADPRFVPCHAVVIENLTHYRPEETRTRRENRQIMEWASSKVKKYLSEICQLHGLHLREVSAAYTSHQDSRTGAPGIRCQDVSLIEFMKSPFWRKQVAQAEKKQKEGKGDAVERYLCELNQKWKGASEEEWRKAGFVRIPLRGGEIFVSAAGHDSPAAKGIHADLNAAANIGLRALLDPDWSGKWWYVPCNSSTMCPARDKVTGSAAVNPGQPLQVSAQLESDDAAKDTKKRKKKGDGKSKEIINLWRDISSYPLEDTRGGTWSNKTVYWNRVQSNVVHILQNQMKG

SEQ ID NO: 6 - protein, 1374 aa, unidentified
MPETTQRAYTLRLQGHDPKDASWREALWKTHEAVNRGAKAFGDWLLTLRGGLDHSLADEGAPGQTPTEEQRKQRRILLALSWLSVESENGAPQEYIVPHDRDNESGARQNWKTREALREILKNRGCRDDEIESWCHDCEPSLTSAIRKDAVWVNRSKAFDNAVQSIPNFSREEIWDLLGCFFVSSQAYLAPLESPKDDKPDASKKDSSKDLIQSAGQWLSRRFGRGKGLNFARLAETYEAIARWASVANPGDTNDLIADLAKTLNAETPELDGILKVVSGPGHKSKTRNLLRSLSAVNHITKDTLQRLKDTANEDAKKAKLKKGEKGHRAYAYKVLEAVEDACGFTYLQEGDRAKHCEFAVMLDHAARRVSSLHTWIKRAEAERRRFEIDTKKKDQLPPSVKEWLDTYCQKRSKETGAVEPYRIRRGAIEGWKEIVEAWSKAGTTTAEDRKHEARRLPDNPHIDKSGDIKLFEDLALEDALPVWHANGDPNNPPDPQLLIDYVEGSEAEFKKRAFKVPTYCHPDPLVHPVFCDYGCSRWNVSFAIQPVKKQKLSSEEKLPAKGLLLDLLHGTAIRPVALRWQSKRFARDLALNTTDSSDKPNEVTRADRFGCALAKCPSSQKIRIRGLFEEKYWNGRLQAPRPELTALAKRVAKYGWDKKARKLRNSLNWFITFSANLRPSGPWEEYTKYAEKAFSSNASAKPSVSRGGFWVVHASPNKRGKMAQLRLCRLPELRVLSVDLGHRYAAACAVWETLSKSAFEQEIHERKILRGGTGPNDLFCHTQHDTNGQSKVTIYRRIGADTLPNGTPHPAPWARLDRQFLIKLPGEEREARKASPTELANVEKLEKELGLKTSENRVKRIDDLMSDTLRTVRQALRRHSLRARIAFNLATLRDQSDGDEESQSKQKRDTRWNNTVKIWHSLLESNEWTDDWAKALWDELGPLSDPQKADDAEWLKLAAEKFYTRWQEDEQTWRERLRWLRRWILPRGSQAASQKGSIRHVGGLSLTRLATIKTLYQVLKAYHMRLKPDNSRKNIPAEGDEALQNFGQKILDDLEHMREQRVKQLASRIVEAALGLGRMKQVTIGKDPKRPREPVDQSCHAVVIENLTHYRPEKRQTRRENRQLMDWSAAKVKKYLKECCQLHGLHLVEVSASYTSRQDSRTGAPGIRCQEVPLTDFLKKNFWREQVKQAKQRLSEGKANARDRYLCQLNERWGNAPAPVTQTAIRLRIPLNGGELFVSADQNSPASKGIQADLNAAANIGLRAITDPDWPGAWWYVPCEANTFKPVKDKVAGSAAIDSNVSLKKDSPNSEKPASDRKSRTSKSMINLWCDTSSKSLSEKDQWQESAPYWEDVAARTINILQASLACSTTNSQ

SEQ ID NO: 7 - protein, 1335 aa, unidentified; identical to SEQ ID NO: 1

SEQ ID NO: 8 - protein, 1520 aa, unidentified
MAYQNGKEQPTVTNQRAYTLRLSGTNDQDSIWRNRLWHTHEAVNKGAKTFGDWLLTMRGGLCHTLAEADVPGKGNKPARHPTPQEIRSRRVVLALSWLSVESQHGAPERHLVSHDLDIATGERKNWKTVEALREILHGRCLCKELIDEWANDCRDSLSATIREDAVWVNRSKAFDLAAKKIGASLTREELWDFLQPFFANKHGYLQMDTVAGVTNGDSETDAEEAKEDSSEEKAKDLSQKAGQWLSSRFGTGTGADFSRFSKVYEVLAARCGSVAVGVSGVEAIRILAGTLADFSPGSNDIEGMLGLMSGPGYKSATRNILQKINTLQTVSQQDLDRLREASEKDALQSKQKVGGKGSRPYANAILQDVEAACGICYAGTGESPARHWQYAVILDHAARRVSMAHSWIKRAEEQRSKFEIEKDKLDHVPKDALAWLDAFCARRSSESGASDAYRIRRSAVDGWKQVVAAWAALPPKPENQGSELLSDAESARIQAARELQDTVEKFGDIQLFEALSLTGAKCVWQPDGRPDAQPLLDYVAGTDAISKKQRFKVPAYRHPDALLHPVFCDFGNSRWNINYAIHRAPEKLTPAQQLLEKKKAEIDKAELTLAKAGDAAKQANISEKINGLRAAFIQQQEKVAWLNSRHAMTMSLWDGTHIEDTPLIWQSKRFGSDIGQPVEAQPLPVSRADRFGRAVALAQDNVPVIPSGLFDLSDWNGRLQAPRRQLEAIAAIRDSAKLSVNEKQQLVAKRIQSIRWLLTFSAKLQSHGPFIAYAAQHGFDWRYGAHGPENKSRQGLAKLILCRLPGLRILSVDLGHRYAAACAVWETLNAGQIQKACLDAGKEAPGPCTLYLHLKQIANGKEKKTIFRRIAADTLPDGSPHPAPWARLDRQFLIKLQGEDRDARLATSEEIAAVEQMENELGVVRQLKRKGRELLVDELMSDALRTLRLGLRRHGVRARIAFNLTANKRIRPGGKEEVLDQEGRVLLLTETLLAWYELYTAERWTDEPARELWNRHIQPLLGATILQNTVNQEDTPSAAKRRKLREETSGKLKHVAEEIAKNDSLCRQLHVLWSAQWQTEDVIWRTRLRMMRRWLLPRGVKRNAQLRISIRDVGGLSLTRIASFKSLYQVQKAYQMRPHPEDPRLNIPERGDSRLENFGQRVLDAMERMRENRVKQLASRIAEAALGIGGETGISSKDGSQKKRPTERSSDPRFAPCHAVVIEDLTHYRPDETQTRRENRQLMSWSSSKVKKYLGEACELNGLYLREVSPAYTSRQDSRTGAPGLRCNDVTVVEFNNSPFWRKQVGAAEKNQKEGNKGDARERYLLSIEEGIRGAANDRDIFRIPVKGGEIFVSACITDGGNNAKKNAPPGLQADLNAAANIGLRAIFDPDWEGRWWYIPCDAATLCPDAKKFIGCKAVDPTKPLRVVAEEGAISASGIGSKKSGRKKNAATDGTRIVNLWRDPSGAPIHRDVLRSPEWQDYAGYWNEVQHRVIRNLKTCYEQTSQQEDPFVSQDADKPF

SEQ ID NO: 9 - protein, 1180 aa, unidentified
MKRLAETALADKVKCETNSRPKGERAYANSILHDVESACGFTYRVDKGEQPVPVSDYSHYANDYRWGPANHSEFAVMLDHAARRVSLAHTWIKRAEAERRQFEENAKKIDKVPKVAREWLDSLCAERSIVLGALEPYRIRRRAVDGWKHVVAAWSKSDCKTAQDRITAARLLQEDPEIDKFGDIQLFEALAEDHAVCVWQRDGEAGKTSDPQLLIDYALAAEAEFKKRHFKVPAYRHPEAFWHPVFCDFGQSRWKICFDVHKNRQSRRQRACANRISRKICFDVHKKRQTLRLSLEVWTGSKMLDMPLCWQCKRLARDLALGQDHKKDRSCQVTRADRLGRAVSNVARNQEVQILGLFEQEYWNGRLQAPRPQLEALGRYIEKHGWDAKAQKSCRAIRWMISFSPRLQPAGPWGKFAEKLQLNPNPKYWPHAEDNKDRGSRSKLILCRLPGLRVLSVDLGHRYAAACAVWEAVDAEQVKEACQAAGHREPNENDLYLHLKKRTTKQKKGSQGVVEETTIYRRIGADTLPDCTPHPAPWARLDRQFLIRLQGEEDEARAASNEEVWAVHKLEAELGRTIPLIDRLLGAGWGQTEKQKARLKALRELGWTPANKCQAFNSTDETELRRPSLAVDELMLDAVGTLRLALKRHGDRARIARYLITDERTKPGGVKEKLDENGRIELLQDALIIWHGLFSSPRWRDDAAKQLWNEHIAKLVGEQNLVEVSEDASGSERRTKQKQNREKLREAAKALVDDVALRQALHDMWKRRWEEEDREWRRRLRWFKDWVLPRREQARKAYSRPAETGSSSHPKRRARYAAIRRVGGLSLTRLATLTEFRRKVQVGFFTRLKPDGTKAEAKEGFGQSTLDALEHLRAQRVKQLASRIVEAALGVGRIRRFPGVKNPKRPDTPVDKPCHAIVIENLTHYRPEETRTRRENRQLMTWSSSKIKKYLAEACQLYGLHLREVTAAYTSRQDSRTGAPGLRCQDVPVKEFMRSLFWRKEVAQAEKKLTAGKGSSYERLLCELNQRWKDNSPGDGKRAELLRLPHKGGEIFVSAAPDSPAARGLQADLNAAANIGLRALTDPDWPGKWWHVPCNAVTFRPVEDKVKGSAAVKLDQSLRQVAHPQSKDPGAKKSKEIVNLWCDISSLPLEHREWKLDWEPYPAYWNNVQCRVIRVLQGKV

SEQ ID NO: 10 - protein, 1546 aa, unidentified
MANAKVKTTTRSYTLSLNAPSDTTDRSPLWHRIFRTHYAICCGAREFGKLLLDLRGGLPTSLAQLGEGIAENDRRQTQRGTRRILALGWLSVEDLDHARNDPHRVQDTAPGSPLDQDLAEKILRKILITKGIKSEEEQSNWISDCLPALTANIRPDAVWVNRAESFAQWQRGTQPGAQPPTPEEAQQILFSLCGESLVTLTLPEQPAAAGQKQPDQETSPDPEEQTDRPPAAPSADDEMDPSNASRGIFGDLFGENAEGKRSRSQGKDNFACAVRDFLCANPTPSADAITEFREKQKPREPNPPGPEKYPPEVSTSGAPTAVAKRYRKLLVCAGLWPKSADEDGSSRNSAKTKFADPKEPQKTEIQINALDLIDACNQAAPADDSGTSPKAGRVFAPAWASNIAEKVASATQMPANAKSLNEFKRLMFALAARRFSQTQSWTRRNEAERHMAAARQDAAVARLREIDPDHKAQDWLRGYEQRRADQSGSNGEFRITRRMIGEAEAVFKAWAGTNSAAERELKTVAVQTTAEKFGDAALYSEIARNTAAEAVWRSGSAPEILDQWVKLRKAQSDQQRTRVPRFCHPNAFRHPTWCEFGESSKPGVWYAWNPKSKPRKPEVGGEGDGTRRLWVLLPDFNSGIGQAVPLRWRSKRLSKDLGEALQPSDAPIPRADRVSIAAAGLNLEGANGVPARYRPSLPFSENTKGWNARLQANRTALLHLESKWDAEAATWRDGGRSLLALKWFTTFSPELAMSEGPGRAIHPKLGWNSEPHSDLNRAQKRGGNAKLILSRLPGLRVLSVDLGHRYAAACAVWETLTTEQMNAACQAKNHTQPAESDMYVHLAHPTERVVKSGRKKGQNLIQTTVYRRIAADTLPDGTPHPAPWGRLDRQFLIKLQGEQRPTRAASKNEADLANALFHRLGLRSDADSENKSRAVDKLMARTVRVATLGLKRHARRAKIAYALDPNTKAIPGMGGSSAAFTPGDEPHIHLLTDALFDWQSLATDAKWDDAHARSLWNHHIATLPGGFHLENPTPRDESAHEPSRQRQRSGDDALRATLKPIAEKLSKADRQEVHAAWKKYWGDSDGQSAIVPKVLQGQRGPEKTTPSASASGWHGKIRWITDWIMGKYLEGCTGHAWKHDVGGLSVSRITTMKSLYQLHKAFAMRATPEKPRGAPEKGESNLGAAQGILTAMESMRQQRVKQLASRIAEAALGAGIERRSDNGRELQRPRERVDDPRFAACHAVVVEDLTNYRPDEMQTRRENRQLMQWASSKVKKYLSEACQLHGLYLRGVPAGYTSRQDSRTGAPGVRCGDIPVEELMAAPRWRRQILTAEKTRRENNTGTARDRYILTLDEKYRLLTAEQRKKTPPARIPVKGGDLFVSADPDSPAASGIQADLNAAANIGLKALIDPDWPGRWWYIPCDATTHKPSPERTRGSAAVDCDVPLGPDSTGTPEDRDAKPKKNQRNSKIAGRGQSAIINLWRDPTHLPIKENPSAWCESKKYWNQVEHNVVKVIESKGQKLTQTAEAATGESASSPPIAPTDVPW

SEQ ID NO: 11 - protein, 1285 aa, unidentified
MEEKMLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 12-14 - skipped (3 entries)

SEQ ID NO: 15 - DNA, 12 bp, synthetic construct
  source 1..12: mol_type = other DNA
tttttttttttt

SEQ ID NO: 16 - DNA, 12 bp, synthetic construct
  source 1..12: mol_type = other DNA
cccccccccccc

SEQ ID NO: 17-18 - skipped (2 entries)

SEQ ID NO: 19 - RNA, 20 nt, synthetic construct
  source 1..20: mol_type = other RNA
ccccccccccccccccaaaa

(19) United States
(12) Patent Application Publication
(10) Pub. No.: US 2026/0218278 A1
(43) Pub. Date: Jul. 30, 2026

(54) COLLATERAL CLEAVAGE BY REPORTER PREFERENCE

(71) Applicant: SHERLOCK BIOSCIENCES, INC., Newton, MA (US)

(72) Inventors: Heike Boisvert, Somerville, MA (US); Jennifer PENA, San Diego, CA (US); Leah CARLSON, Newton, MA (US)

(21) Appl. No.: 19/141,830

(22) PCT Filed: Dec. 20, 2023
(86) PCT No.: PCT/US2023/085253

§ 371 Date: Jun. 20, 2025

Related U.S. Application Data
(60) Provisional application No. 63/434,300, filed on Dec. 21, 2022.

(51) Int. Cl. C12Q 1/683; G01N 33/53

(52) U.S. Cl. CPC C12Q 1/683; G01N 33/5308

(57) ABSTRACT

The present invention provides use of Cas enzymes and methods and kits for detecting a target nucleic acid. The provided technologies utilize preferential collateral cleavage specificity for delectably labeled nucleic acid probes of certain Cas enzymes.

### BACKGROUND

It is increasingly important that technologies are developed that can specifically detect nucleic acid of interest in a sample and in particular more than two nucleic acids of interest in a single sample (e.g., vessel).

### SUMMARY

The present disclosure describes technologies (e.g., compositions and methods) that permit detection of target nucleic acids (e.g., a plurality of target nucleic acids) present in a single sample. In some embodiments, provided technologies utilize nucleic acid cleavage activity of certain Cas enzymes; in many embodiments, provided technologies utilize collateral cleavage activity of certain Cas enzymes; in some embodiments, provided technologies utilize preferential collateral cleavage specificity for detectably labeled nucleic acid probes of certain Cas enzymes (e.g., Cas enzymes having orthogonal trans-cleavage preference for certain detectably labeled nucleic acid probes). The present disclosure identifies the source of a problem associated with certain previously-developed technologies that utilize Cas collateral cleavage activity of certain detectably labeled nucleic acid probes when used to detect a plurality of target nucleic acids in a single sample. The present disclosure solves such problems and, moreover, provides technologies with unexpected benefits and/or capabilities relative to alternative available systems.

The present disclosure provides a surprising insight that Cas enzymes having collateral activity display preferential cleavage specificity for a corresponding detectably labeled nucleic acid probe comprising a reporter dye. Interestingly, the inventors found new Cas enzyme groups characterized by having high collateral activity to a corresponding detectably labeled nucleic acid probe comprising a reporter dye while demonstrating low or no activity to non-corresponding detectably labeled nucleic acid probes having other reporter dyes. The disclosure thus provides orthogonal Cas enzyme/probe pairs wherein the Cas enzyme demonstrates preferential collateral cleavage activity to its corresponding detectably labeled nucleic acid probe, as well as two Cas enzyme/probe pairs, which are mutually orthogonal and active in the same system (e.g., a single vessel or a single cell). The present disclosure allows for detection of multiple target nucleotide sequences by activation of different reporter dyes (e.g., detection of different reporter dye signals).

The present disclosure provides robust multiplexed CRISPR-based detecting using two Cas enzymes (e.g., a first and a second Cas enzyme) demonstrating preferential cleavage specificity for their corresponding detectably labeled nucleic acid probe having different reporter dyes to simultaneous detect two different target nucleotide sequences.

### BRIEF DESCRIPTION OF THE DRAWING

FIGS. 1A-C show cleavage of (A) C7-FAM probe; (B) C7-TxRed probe; and (C) C7-Cy5 probe by PAL5 or RS9. FIGS. 1A-C demonstrate PAL5 relative specificity for cleaving a C7-FAM probe, but not C7-probes with other reporter dyes. O: SARS-COV-2 Orflab gene. N: SARS-COV-2 N-gene; and RP: RnaseP.

FIGS. 2A-B show (A) the components of multiplex real-time SHERLOCK (SLK) using SLK9 Cas12a (RS9; SEQ ID NO: 11) and AacCas12b; and (B) ANS/saline samples tested with multiplexed, real-time SLK SLK9/Aac assay. Fluorescence values greater than background values are reported in time to result (TTR) and were determined for red channel fluorescence (SARS-COV-2) and green channel fluorescence (Rnase P control). Samples that do not show increased fluorescence above the cut off value are plotted in the ND (not determined) zone. ANS: Anterior nasal swab. SLK: real-time SHERLOCK (loop-mediated isothermal amplification (LAMP) combined with real-time, single-step nucleic acid detection method).

FIGS. 3A-B show (A) the components of multiplex real-time SHERLOCK (SLK) using SLK9 Cas12a (RS9; SEQ ID NO: 11) and SLK5-2 Cas12b (PAL5; SEQ ID NO: 5); and (B) ANS/Tris-EDTA samples tested with multiplexed, real-time SLK SLK9/Aac assay. Fluorescence values greater than background values are reported in time to result (TTR) and were determined for red channel fluorescence (SARS-COV-2) and green channel fluorescence (RnaseP control). Samples that do not show increased fluorescence above the cut off value are plotted in the ND (not determined) zone. ANS: Anterior nasal swab. SLK: real-time SHERLOCK (loop-mediated isothermal amplification (LAMP) combined with real-time, single-step nucleic acid detection method).

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell). Binding between two entities may be considered “specific” if, under the conditions assessed, the relevant entities are more likely to associate with one another than with other available binding partners.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate includes predominantly hydrophilic components; in some embodiments, a cellular lysate includes predominantly hydrophobic components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells; in some embodiments, such a lysate is referred to as a “primary” lysate. In some embodiments, one or more isolation or purification steps is performed on a primary lysate; however, the term “lysate” refers to a preparation that includes multiple cellular components and not to pure preparations of any individual component.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Detectable entity: The term “detectable entity” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., 3H, 14C, 18F, 19F, 32P, 35S, 135I, 125I, 123I, 64Cu, 187Re, 111 In, 90Y, 99mTc, 177Lu, 89Zr etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, dioxigenin, haptens, and proteins for which antisera or monoclonal antibodies are available.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Homology: As used herein, the term “homology” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially homologous” to one another if their sequences are at least 25%, 30%, 35%, 40%, 45%, 50%, 55%, 60%, 65%, 70%, 75%, 80%, 85%, 90%, 95%, or 99% homologous, meaning that identical or homologous residues are present in corresponding positions of both molecules. Calculation of percent homology of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; residues at corresponding positions are then compared. When a position in the first sequence is occupied by the same residue (e.g., nucleotide or amino acid) as a corresponding position in the second sequence, then the two molecules (i.e., first and second) are identical at that position. When a position in the first sequence is occupied by the same residue or by a structurally and/or functionally related residue (as will be understood by those skilled in the art, in context), then the two molecules are considered “homologous” at that position. Percent homology between two sequences is a function of the number of homologous positions shared by the two sequences being compared, taking into account the number of gaps, and the length of each gap, which needs to be introduced for optimal alignment of the two sequences. Comparison of sequences and determination of percent homology between two sequences can be accomplished using a mathematical algorithm. For example, percent homology between two nucleotide sequences can be determined using the algorithm of Meyers and Miller (CABIOS, 1989, 4:11-17, which is herein incorporated by reference in its entirety), which has been incorporated into the ALIGN program (version 2.0).

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; residues at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4:11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel, in cell culture, etc., rather than within a multi-cellular organism.

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. Those skilled in the art will appreciate that, in many embodiments, a useful marker need not distinguish with 100% accuracy.

Mutant: As used herein, the term “mutant” refers to an entity that shows significant structural identity with a reference entity but differs structurally from the reference entity in the presence or level of one or more chemical moieties as compared with the reference entity. In many embodiments, a mutant also differs functionally from its reference entity. In general, whether a particular entity is properly considered to be a “mutant” of a reference entity is based on its degree of structural identity with the reference entity. As will be appreciated by those skilled in the art, any biological or chemical reference entity has certain characteristic structural elements. A mutant, by definition, is a distinct chemical entity that shares one or more such characteristic structural elements. To give but a few examples, a small molecule may have a characteristic core structural element (e.g., a macrocycle core) and/or one or more characteristic pendent moieties so that a mutant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a mutant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a mutant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a mutant polypeptide does not share at least one characteristic sequence element with a reference polypeptide. In some embodiments, the reference polypeptide has one or more biological activities. In some embodiments, a mutant polypeptide shares one or more of the biological activities of the reference polypeptide. In some embodiments, a mutant polypeptide lacks one or more of the biological activities of the reference polypeptide. In some embodiments, a mutant polypeptide shows a reduced level of one or more biological activities as compared with the reference polypeptide.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Orthogonal: As used herein, the term “orthogonal” refers to the ability of a Cas enzyme or detectably labeled nucleic acid probe to interact with a specific corresponding detectably labeled nucleic acid probe or a specific corresponding Cas enzyme, respectively, without any cross-reactivity with other Cas enzymes or detectably labeled nucleic acid probes, such as endogenous Cas enzymes or detectably labeled nucleic acid probes or Cas enzymes or detectably labeled nucleic acid probes present in the same system as the Cas enzymes or detectably labeled nucleic acid probes of interest. An orthogonal Cas enzyme can only cleave its cognate corresponding detectably labeled nucleic acid probe, whereas a non-corresponding Cas enzyme is not capable of cleaving the cognate corresponding detectably labeled nucleic acid probe. A Cas enzyme/detectably labeled nucleic acid probe pair is an orthogonal pair, if the pair does not demonstrate cross-reactivity with other Cas enzymes or detectably labeled nucleic acid probes, while still being functional compatible with a guide polynucleotide.

Predetermined: By predetermined is meant deliberately selected, for example as opposed to randomly occurring or achieved.

Preferential cleavage: The term “preferential cleavage”, when used herein with reference to an agent having a cleavage activity, is understood by those skilled in the art to mean that the agent discriminates between potential target entities. For example, in some embodiments, an agent is said to preferential cleavage a target entity if it preferentially cleavages that target in the presence of one or more competing alternative targets.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Single Nucleotide Polymorphism (SNP): As used herein, the term “single nucleotide polymorphism” or “SNP” refers to a particular base position in the genome where alternative bases are known to distinguish one allele from another. In some embodiments, one or a few SNPs and/or CNPs is/are sufficient to distinguish complex genetic variants from one another so that, for analytical purposes, one or a set of SNPs and/or CNPs may be considered to be characteristic of a particular variant, trait, cell type, individual, species, etc, or set thereof. In some embodiments, one or a set of SNPs and/or CNPs may be considered to define a particular variant, trait, cell type, individual, species, etc, or set thereof.

Specific: The term “specific”, when used herein with reference to an agent having an activity, is understood by those skilled in the art to mean that the agent discriminates between potential target entities or states. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., a reporter dye, an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree and/or rate of association between the binding agent and its partner; in some embodiments, specific binding is assessed by detecting or determining degree and/or rate of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specificity: As is known in the art, “specificity” is a measure of the ability of a particular ligand to distinguish its binding partner from other potential binding partners.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

### DETAILED DESCRIPTION OF CERTAIN EMBODIMENTS

In some embodiments, the present disclosure provides technologies that utilize a first Cas enzyme having collateral cleavage activity and a first detectably labeled nucleic acid probe comprising a reporter dye, wherein the first Cas enzyme demonstrates preferential cleavage specificity for its corresponding first detectably labeled nucleic acid probe. In some embodiments, the present disclosure further provides technologies that utilize a second Cas enzyme having collateral cleavage activity and a second detectably labeled nucleic acid probe comprising a reporter dye that is different from the first reporter dye, wherein the second Cas enzyme demonstrates preferential cleavage specificity for its corresponding second detectably labeled nucleic acid probe. Provided technologies are useful in real-time, multiplexed CRISPR-based diagnostics. A first and/or a second Cas enzyme can be programmed to detect separate target sequences.

### Compositions

In some embodiments, the present disclosure provides compositions (e.g., non-naturally occurring or engineered compositions) comprising at least one Cas enzyme and a corresponding detectably labeled nucleic acid probe (e.g., a first Cas enzyme and a first detectably labeled nucleic acid probe). In some such embodiments, the first detectably labeled probe is orthogonal to the first Cas enzyme.

In some embodiments, the present disclosure provides compositions (e.g., non-naturally occurring or engineered compositions) comprising two Cas enzymes and two detectably labeled nucleic acid probes (e.g., a first and a second Cas enzyme and a first and a second detectably labeled nucleic acid probe). In some such embodiments, the two Cas enzymes and their corresponding detectably labeled nucleic acid probes are mutually orthogonal. In some embodiments, the present disclosure provides compositions (e.g., non-naturally occurring or engineered compositions) comprising at least two Cas enzymes and at least two detectably labeled nucleic acid probes (e.g., at least a first and at least a second Cas enzyme and at least a first and at least a second detectably labeled nucleic acid probe).

In some embodiments, the present disclosure provides compositions (e.g., non-naturally occurring or engineered compositions) comprising a first Cas enzyme having collateral cleavage activity and a first detectably labeled nucleic acid probe comprising a first reporter dye and a second Cas enzyme having collateral cleavage activity and a second detectably labeled nucleic acid probe comprising a second reporter dye, wherein the first and second Cas enzyme are different and the first and second detectably labeled nucleic acid probe are different, and wherein both the first and second Cas enzyme demonstrate preferential cleavage specificity for its corresponding first or second detectably labeled nucleic acid probe compared to its non-corresponding detectably labeled nucleic acid probe.

### Cas Enzymes

Cas enzymes were originally identified as part of the CRISPR (which stands for “clusters of regularly interspaced short palindromic repeats”)-Cas (which stands for “CRISPR-associated”) systems that provide microbes with adaptive immunity to infectious nucleic acids. Those of ordinary skill in the art are aware of an enormous number of Cas enzymes, and of sequence elements and functional characteristics that categorize them into different classes. Class 1 CRISPR-Cas systems have multi-subunit effector complexes; Class 2 systems have single-subunit effectors.

At least six different “Types” of Cas proteins have been described; Types I, II, and IV are Class 1 enzymes whereas Types II (including Cas9), V (including Cas 12 and Cas 14), and VI (including Cas 13) are Class 2 enzymes. Technologies for identifying Cas enzymes, and classifying them (e.g., based on presence, organization, and/or sequence of a RuvC domain and/or one or more other sequence elements) are by now well known in the art. Moreover, many Cas variants have been prepared, and those of ordinary skill in the art have a good understanding of structural (e.g., sequence) elements that participate in (e.g., are necessary and/or sufficient for) activities of Cas enzymes.

In some embodiments, a Cas enzyme of the present disclosure has collateral cleavage activity (e.g., cleaves non-target nucleic acid when its guide polynucleotide binds to its target and the Cas enzyme forms a complex herewith). In some embodiments, a Cas enzyme cleaves a detectably labeled nucleic acid probe (e.g., comprising a nucleic acid sequence and a reporter dye). In some embodiments, present technologies utilize a Cas enzyme having collateral activity, and detects activation of that collateral cleavage activity by measuring cleavage of a detectably labeled nucleic acid probe.

The inventors surprisingly identified new Cas enzyme groups demonstrating differential detectably labeled nucleic acid probe cleavage specificity. In some such embodiments, Cas enzymes within a class are characterized by demonstrating preferential cleavage specificity for a particular detectably labeled nucleic acid probe (e.g., a corresponding detectably labeled nucleic acid probe), whereas they do not cleave other detectably labeled nucleic acids probes (non-corresponding probes). In some embodiments, a Cas enzyme having collateral cleavage activity demonstrates preferential cleavage specificity for a particular detectably labeled nucleic acid probe. In some embodiments, a Cas enzyme only cleaves its corresponding detectably labeled nucleic acid probe. In some embodiments, a Cas enzyme does not cleave a non-corresponding detectably labeled nucleic acid probe.

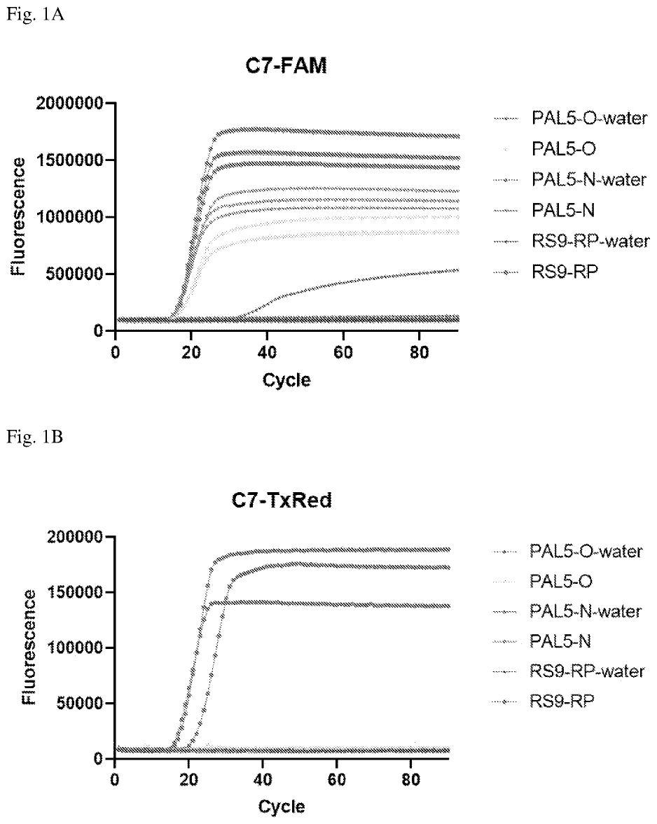

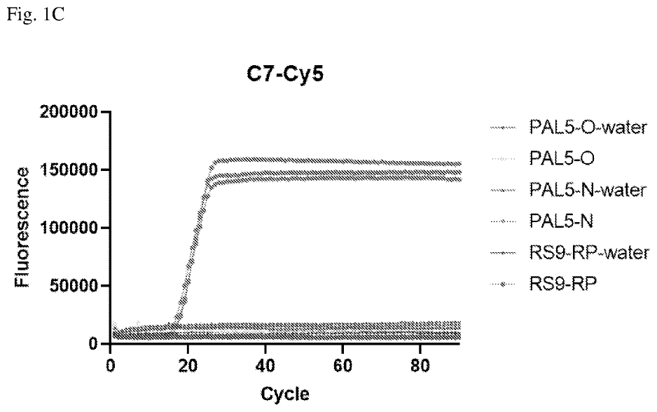

Without wishing to be bound by any particular theory, it is proposed that Cas enzymes demonstrate orthogonal cleavage preference of bystander detectably labeled nucleic acid probes based on their reporter dye, sequence length, type of nucleic acids, or a combination thereof. The present disclosure shows that Cas enzymes demonstrate orthogonal cleavage preference of probes by recognizing the reporter dye (FIGS. 1A-C). In some embodiments, Cas enzymes do not cleave a nucleic acid probe sequence by recognizing the nucleic acids within the sequence, but cleave the nucleic acid probe sequence by recognizing the reporter dye. In some embodiments, Cas enzymes cleave a detectably labeled nucleic acid probe by recognizing the detectably labeled nucleic acid probe sequence (e.g., length, type of nucleic acids, or a combination thereof) (Table 4). In some embodiments, Cas enzymes cleave a detectably labeled nucleic acid probe by recognizing the reporter dye, the length, the type of nucleic acid residues, or a combination thereof.

In some embodiments, a Cas enzyme is characterized in that it preferentially cleaves its corresponding detectably labeled nucleic acid probe comprising a reporter dye; in some such embodiments, such Cas enzyme is characterized in that no detectable non-corresponding detectably labeled nucleic acid probe cleavage is detected under conditions where robust corresponding detectably labeled nucleic acid probe cleavage is observed. In some embodiments, a Cas enzyme cleaves its corresponding detectably labeled nucleic acid probe having a reporter dye without any cross-reactivity with an otherwise comparable non-corresponding detectably labeled nucleic acid probe having a different reporter dye. In some embodiments, a Cas enzyme cleaves its corresponding detectably labeled nucleic acid probe comprising a reporter dye with a specificity that is at least 2 fold higher than its specificity for a non-corresponding detectably labeled nucleic acid probe having a different reporter dye. In some embodiments, a Cas enzyme cleaves its corresponding detectably labeled nucleic acid probe having a reporter dye with a specificity that is at least 3 fold higher, such as at least 4 fold higher, such as at least 5 fold higher, such as at least 10 fold higher, such as at least 15 fold higher, such as at least 20 fold higher, such as at least 50 fold higher, such as at least 100 fold higher, such as at least 500 fold higher than its specificity for a non-corresponding detectably labeled nucleic acid probe having a different reporter dye.

In some embodiments, a first Cas enzyme demonstrates preferential cleavage for its corresponding first detectably labeled nucleic acid probe comprising a reporter dye compared to a non-corresponding detectably labeled nucleic acid probe comprising a different reporter dye (e.g., a second detectably labeled nucleic acid probe). In some embodiments, a first Case enzyme does not demonstrate any cleavage of a non-corresponding detectably labeled nucleic acid probe (e.g., a second detectably labeled nucleic acid probe).

In some embodiments, a second Cas enzyme demonstrates preferential cleavage for its corresponding second detectably labeled nucleic acid probe comprising a reporter dye compared to a non-corresponding detectably labeled nucleic acid probe comprising a different reporter dye (e.g., a first detectably labeled nucleic acid probe). In some embodiments, a second Case enzyme does not demonstrate any cleavage of a non-corresponding detectably labeled nucleic acid probe (e.g., a first detectably labeled nucleic acid probe).

The present disclosure provides Cas enzymes that cleave a detectably labeled nucleic acid probe characterized by having a particular reporter dye with preferential specificity relative to an otherwise comparable detectably labeled nucleic acid probe characterized by having a different reporter dye. In some embodiments, a Cas enzyme demonstrates preferential cleavage specificity for a detectably labeled nucleic acid probe characterized by having a particular reporter dye. Thus, this disclosure provides Cas enzymes demonstrating preferential cleavage specificity for probes having a particular reporter dye. In some embodiments, such specificity is not affected by the nucleic acid probe sequence (e.g., sequence length and/or nucleic acid type). In some embodiments, such specificity is not affected by the nucleic acid probe length.

In some embodiments, reporter dye preference can be determined by assessing Cas enzyme collateral activity by comparing collateral cleavage activity when contacting a Cas enzyme with a detectably labeled nucleic acid having a nucleic acid sequence and reporter dye compared to a detectably labeled nucleic acid having the same nucleic acid sequence but a different reporter dye.

The present disclosure further provides Cas enzymes that cleave a detectably labeled nucleic acid probe characterized by having a particular detectably labeled nucleic acid probe length with preferential specificity relative to an otherwise comparable detectably labeled nucleic acid probe characterized by having a different detectably labeled nucleic acid probe length. In some embodiments, a Cas enzyme demonstrates preferential cleavage specificity for a detectably labeled nucleic acid probe characterized by having a particular detectably labeled nucleic acid probe length. This disclosure additionally provides Cas enzymes demonstrating preferential cleavage specificity for probes having a particular detectably labeled nucleic acid probe length. In some embodiments, the present disclosure provides Cas enzymes demonstrating preferential cleavage specificity for a detectably labeled nucleic acid probe having a particular length.

In some embodiments, detectably labeled nucleic acid probe length preference can be determined by assessing Cas enzyme collateral activity by comparing collateral cleavage activity when contacting a Cas enzyme with a detectably labeled nucleic acid having a specific type of nucleic acids and reporter dye compared to a detectably labeled nucleic acid having the same type of nucleic acids and reporter dye, but different detectably labeled nucleic acid probe length.

The present disclosure further provides Cas enzymes that cleave a detectably labeled nucleic acid probe characterized by having a particular type of nucleic acids with preferential specificity relative to an otherwise comparable detectably labeled nucleic acid probe characterized by having a different type of nucleic acids. In some embodiments, a Cas enzyme demonstrates preferential cleavage specificity for a detectably labeled nucleic acid probe characterized by having a particular type of nucleic acids. This disclosure additionally provides Cas enzymes demonstrating preferential cleavage specificity for probes having a particular type of nucleic acids.

In some such embodiments, detectably labeled nucleic acid probe type preference can be determined by assessing Cas enzyme collateral activity by comparing collateral cleavage activity when contacting a Cas enzyme with a detectably labeled nucleic acid having a specific length and reporter dye compared to a detectably labeled nucleic acid having the same length and reporter dye, but different nucleotides.

In some embodiments, Cas enzymes as provided herein demonstrate preferential cleavage specificity for a detectably labeled nucleic acid probe by recognizing both the reporter dye and the nucleic acid probe sequence (e.g., length and/or nucleic acid type). In some embodiments, a Cas enzyme demonstrates preferential cleavage specificity for a detectably labeled nucleic acid probe having a particular reporter dye and a particular sequence length. In some embodiments, a Cas enzyme demonstrates preferential cleavage specificity for a detectably labeled nucleic acid probe having a particular reporter dye and particular probe nucleotide sequence.

In some embodiments, a Cas enzyme as provided herein demonstrate preferential cleavage specificity for a detectably labeled nucleic acid probe by recognizing the nucleic acid probe sequence (e.g., length and/or nucleic acid type). In some embodiments, a Cas enzyme demonstrates preferential cleavage specificity for a detectably labeled nucleic acid probe having a particular sequence length, particular type of nucleic acids, or a combination thereof.

In some embodiments, a first Cas enzyme demonstrates preferential cleavage for its corresponding first detectably labeled nucleic acid probe comprising a first sequence length compared to a non-corresponding detectably labeled nucleic acid probe having a different sequence length (e.g., a second detectably labeled nucleic acid probe).

In some embodiments, a first Cas enzyme demonstrates preferential cleavage for its corresponding first detectably labeled nucleic acid probe comprising a first sequence having a particular type of nucleic acids compared to a non-corresponding detectably labeled nucleic acid probe comprising a sequence having different nucleic acids (e.g., a second detectably labeled nucleic acid probe).

In some embodiments, a Cas enzyme as provided herein demonstrates collateral cleavage preference of an RNA or a DNA detectably labeled nucleic acid probe having a reporter dye. In some such embodiments, RNA or DNA preference can be characterized by assessing Cas enzyme collateral activity by comparing collateral cleavage activity when contacting a Cas enzyme with a DNA detectably labeled nucleic acid probe having a reporter dye compared to an RNA detectably labeled nucleic acid probe having the same reporter dye.

In some embodiments, a Cas enzyme as provided herein demonstrates preferential cleavage specificity for a corresponding detectably labeled nucleic acid comprising a reporter dye, wherein the probe is further characterized by having a particular sequence length. Without wishing to be bound by any specific theory, it is proposed that a sequence length of at least 7 nucleotides allows the reporter dye to be free of any probe stereo chemical hindrance, hereby allowing the Cas enzyme to recognize the particular reporter dye.

In some embodiments, a detectably labeled nucleic acid probe comprising a reporter dye, has a sequence length of at least 7 nucleotides, at least 8 nucleotides, at least 9 nucleotides, at least 10 nucleotides, at least 11 nucleotides or at least 12 nucleotides. In some embodiments, a Cas enzyme as provided herein demonstrates preferential cleavage specificity for a corresponding detectably labeled nucleic acid probe comprising a reporter dye, having a sequence length of about 7 nucleotides or about 12 nucleotides.

In some embodiments, collateral cleavage activity of a first Cas enzyme and first guide polynucleotide or a second Cas enzyme and a second guide polynucleotide when contacted with a sample comprising a target nucleic acid, collateral cleavage activity of the first Cas enzyme or the second Cas enzyme is increased relative to an appropriate reference standard. In some embodiments, an appropriate reference standard is or comprises a sample that does not comprise a target nucleic acid. In some embodiments, an appropriate reference standard comprises a thermostable Cas enzyme with collateral activity further comprising a mutation that abolishes the collateral cleavage activity.

In some embodiments, a Cas enzyme according to the present disclosure is a thermostable Cas enzyme. In some embodiments, a thermostable Cas enzyme performs (e.g., its collateral cleavage activity functions sufficiently) at temperatures above about 50° C.; in some embodiments, above a temperature selected from the group consisting of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In some embodiments, useful thermostable Cas enzymes perform (e.g., its collateral cleavage activity functions sufficiently) at temperatures above about 60° C.

In some embodiments, a thermostable Cas Protein performs (e.g., its collateral cleavage activity functions sufficiently) within a temperature range at which nucleic acid extension and/or amplification reaction(s) are performed; those skilled in the art are well familiar with various such reactions and the temperature ranges at which they are performed. In some embodiments, a temperature range may be above a temperature selected from the group consisting of about 60° C., about 61° C., about 62° C., about 63° C., about 64° C., 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In some embodiments, a temperature range may be about 60° C. to about 90° C. In some embodiments, a temperature range may be about 60° C. to about 80° C. In some embodiments, a temperature range may be about 60° C. to about 75° C. In some embodiments, a temperature range may be about 65° C. to about 90° C. In some embodiments, a temperature range may be about 60° C. to about 80° C. In some embodiments, a temperature range may be about 60° C. to about 75° C.

In some embodiments, a Cas enzyme demonstrating preferential collateral cleavage activity as provided herein is a Cas12 (e.g., Cas12a or Cas12b) enzyme. In some embodiments, a Cas enzyme with preferential collateral cleavage activity as provided herein is a Cas enzyme comprising an amino acid sequence having at last 80%, at least 85%, at least 90%, at least 91%, at least 92%, at least 93%, at least 94%, at least 95%, at least 96%, at least 97%, at least 98% at least 99% or 100% sequence identity to any one of SEQ ID NOs: 1-11. In some embodiments, a Cas enzyme comprises an amino acid sequence selected from the group consisting of SEQ ID NO: 5, SEQ ID NO: 9, SEQ ID NO: 10 and SEQ ID NO: 11. In some embodiments, a first Cas enzyme comprises an amino acid sequence of SEQ ID NO: 11 and a second Cas enzyme comprises an amino acid sequence of any one of SEQ ID NO: 5, SEQ ID NO: 9, and SEQ ID NO: 10.

TABLE 1
Cas enzyme amino acid sequence

| SEQ ID | | |
|---|---|---|
| NO: | Enzyme | Amino Acid Sequence |
| 1 | PAL001 | MKSLAQFQNLYALQKTLRFELKPEGHTRETFNRWLEEIEKE |
| | (Cas12a) | QASENENIVYQDLLRAKKYEKIKIILDEYHKDFIEQALAYA |
| | | NLTELEKYEELYRKSNRTSEEEEEFENTKESLRKQIANIFIKN |
| | | PNKTVQERWKFLFSKKLIQNELIVWVKGNYELLSEKLKNEF |
| | | PDESSIISTIEDFKYFTTYFRNYHENRKNLYSNEDKFSTIAHR |
| | | LIHENLPKFIDNIAIYQKAKAVLNINEVEKELGLPEDTLDKIF |
| | | SLDFFSKALTQKGIDQYNYFLGGKTENEVKKIKGLNEFINL |
| | | YNQQQQDKNQRLPFLKVLYKLPLFERTSTSFRFEPIENDRD |
| | | LIERIGKFYYNDLKQYRDDSQGDTTDILSGINTLLRHVHDY |
| | | RDGLYVNGGITLTQISQKIFGSWSYINNALAYFYDTYIDASG |
| | | VDHQGERKPKKQKQIQEKTKWLKQKQFPVILVEKALSEYK |
| | | SIETNEDLKTRISDTTLCDFFKRCGNDDNGQDLFDRIEARLR |
| | | EKNEEGYSLEDLLKKEFTTERKLMQDKTKTLLIKNFLDVIQ |
| | | GDKDDITAGLLHFVKCLIPRTEISEKNELFYSGMEKYLNILS |
| | | EVTPLYNKARNYLTQKPYSIEKVKLNFENSTLLDGWDENE |
| | | ESDNSCVLLRKRGYYYLGIMNKKHNMIFDRKIYPKATEGE |
| | | AYYEKMIYKLLPGAYKMLPKVFFSEKNIDYFKPSEEILRIRN |
| | | TASYSKNGQPQEGYQKASFSIEDCRKYIDFFKKCIANHWD |
| | | WQKFNFNFSPTEYYQSIDEFYREIERQGYKIDFVKIPESYINQ |
| | | LIKENKLYLFKIYNKDFSEKKKSKGKDNLHTLYWKMLFDE |
| | | KNLKDVVLKLNGEAEVFFRQKSILYNEEIWNKGHHYSELK |
| | | DRFSYPIISNKRYAEDKFFLHVPITLNFKADGINNVNNMVN |
| | | EFIKDNRDIHIIGIDRGERHLLYVSVINQKGDIVEQCSLNEIV |
| | | TEYNGKIFKKNYHEELDNLEKERDRARKDWQTIANIKELK |
| | | EGYLSHVIHKISKLILKYNAIVVMEDLNSGFKRGRQKVEKQ |
| | | VYQNFEKQLIEKLNYLVLKESNVDEPGGVLRAYQLANKFE |
| | | TFKKLGKQSGIIFYVPAAYTSAIDPVTGYIQYLYPLKQADSV |
| | | EKARKFYSQFKRISYNPHKQWFEFSFDYNDFNIIYHGKSSW |
| | | TICTTNTERYMWNRLLNNGHGGEELVYVTNELELLFGEYN |
| | | IIYGDGKDIKQQITDVQDIDVDRTAKQFYKRINELLNLTLKL |
| | | RHNNGKKGADEEDYILSPVEPYFDSRFESRKPSMQQTLPIN |
| | | ADANGAFNIARKGLLLLERLNQLGVEEFEKTKKSNNKKTQ |
| | | WLPHELWVEYAQNHTRK |
| 2 | PAL002 | MQDKTGWSSFTNKYSLSKTLRFELKPVGNTQKMLEDDGV |
| | (Cas12a) | FQKDRERQENYKKVKPFMDKLHREFIKEALNNLKLEGLTE |
| | | YFEIFKKFRKDKNNKELKNAEKKLRQIIGRCYTETAQIWVE |
| | | KYKEFGFKKKNIGFLFEEGVFELMKLKYGNDEASQIEKNGE |
| | | VLSIFDGWKGFLGYFKKFFETRNNFYKDDGTSTAVSTRIINE |
| | | NLKIYLDNLIKYNKIKDKVDFKEADILQENKLNLSDFFNVE |
| | | SYAKYSLQKGIDYYNEILGGKTLKNGTKLKGLNEVINEYK |
| | | QKNKSGELSKFKMLKKQILGEGEDRTLFEEIENEDELKDVL |
| | | KDFFYNADPKITLFKTLLEDFFSNTEKYKDELDKIYFNTVAI |
| | | NGILHRWVDDSGVFQKYLFEVLKSNKLVKSNHYDKKEDS |
| | | YKFPDFISFEHIKVALENCERDGLKDKFWKEKYYTKECLTE |
| | | NGLANLWQEFLEIYKCEFKKLYDYKTDDNDCYLQYRDNY |
| | | KKYILDANFNPKEKSAKDIIKDYLDSVLSIYQLAKYFALEK |
| | | KKVWTTDYETGDFYYEYIKFYEDTYEQIIKPYNLVRNYLTR |
| | | KPINTAKKWKLNFDNAYLASGWDKDKEVSNLTVILRRDEQ |
| | | YYLAIMKKGKNKIFEKKFSCGEFEKMEYKQIAEASSDIHNL |
| | | VLMNDGSCRRCIKMHDKRKYWPLDISIIKEKKSYAKENFV |
| | | RRDFERFVNYMKKCSLLYWKEYDLKFSDTSTYKNINDFTN |
| | | EIASQGYKLSFSAIPESYINEKNNNGELYLFQIYNKDFGIKTE |
| | | GNKNLHTMYWESIFSEENRFRNFIVKLNGKAEIFYRPKSEQ |
| | | VEKEQRNFTREIIKNRRYTENKIYFHCPITLNRISRENVKKFN |
| | | NGINNYIATNPNINILGVDRGEKHLVYYAIVDQDGKLIDAE |
| | | DATGSFNTIGSTDYHRLLEEKAKDREKERKDWDLIRGIKDL |
| | | KKGYISLVVRKIADLAIKYNAIIIFEDLNTRFKQIRGGMEKS |
| | | VYQQLEKALINKLSFLVNKGEKDPEQAGHLLKAYQLAAPF |
| | | QTFDKMGRQTGIIFYTQASYTSKIDPITGWRPNLYLKYRNID |
| | | DSKESIKKFKSILFNKEKNRFEFTYDLKDFVDFEEDKIPEKT |
| | | EWTLCSSVERHKWNRHMNNNKGGYEVYKDLTENFYKLF |
| | | DENNISMNKDIVDQVESISNGNFFRQFIYLFNLVCQIRNTDE |
| | | KAEDVDKRDFILSPVEPFFDSRRAKDFKAYGDNLPKNGDE |
| | | NGAYNIARKGVLIIKKIKEYYNQNGSCDKLGWGDLSISHKE |
| | | WDDFATNN |
| 3 | PAL003 | MDSYEQFTKLYPIQKTIRFELKPQGRTKEHFDNSNFLEKDR |
| | (Cas12a) | ERDDNYKILKEVIDDYHREFIDECLSNIQLNWDDLKKFSEE |
| | | YRRSKEKKNNRDSESEQKRMSTTSETRAINKKNLEAEQKR |
| | | MRGEIVSAFKKDDRFKHLFSEKLFSILLKNQIYEKGTLEEIE |
| | | AFDCFNKFSGYFKSFHENRKNMYSDEDKETAISYRIINENFP |
| | | KLLDNFEKYQYVCREYPEQIREAESTLAEAGCYIKMDEIFSI |
| | | DNFNNVMMQGGKESGISRYNLAIGGIVQGTGEKPKGLNEF |
| | | LNLAYQNEPNGRKKIRMEPLYKQILSKEESFSYRLEAFTDD |
| | | SQLLSAIRSFFDIVEKDKNGNIFDRAVNLMSSFSNYDTSKIYI |
| | | RKAYLNQVSKEIFGYRGKSDSKPAKTADESLNKSGGWEKL |
| | | GQMLRDYKADSIGDRNLEKTCKKVDKWLDSDEFTLSDILG |
| | | AISLAGSNETFEAYVSEICVARRNIDKEKEKEKNINVEKISG |
| | | DTESIQIIKALLDSVQEFFHLLSPFQLHPNTPHDWTFYAEFN |
| | | DIYDKLSAITPLYNQARNHLTKKNLDTSKIKLNFNNPTLAN |
| | | GWDVNKEYENTAVILIRDGKYYLGIMNPKNKRKIKFDEGS |
| | | GAGPFYQKMVYKLLPGPYRMLPKVFFAKKNIDYYNPSQEI |
| | | REGYKAGKHKKGKEFDKGFCHKLIDFFKESIQKNENWKVF |
| | | DFKFSPTESYDDISEFYQEVEKQGYRMYFVNIPSDTIDRYVE |
| | | GGDMFLFQIYNKDFAKGAKGNKDMHTLYWNAVFSEENLQ |
| | | KGVMKLSGEAELFYRKKSDIKDPPHREGEILVNRTYIDRTH |
| | | VSGVMGEQNTVKESRIPVPDEIHKNLFDYYNHGRELTKEE |
| | | KEYCDKVGSFKAYYGIVKDRRYLENKMYFHVPLTLNFKAI |
| | | GEKRINKMAIEKFLTDENACIIGIDRGERNLLYYSIIDRNGKI |
| | | IDQKSLNVIDGFDYHEKLSQRQTEREVARQSWNSIGKIKDL |
| | | KEGYLAKAVHEISKMAIKYNAIVVLEDLHFGFKKGRLKVE |
| | | KQIYQKFEEMLINKLNYLVFKDVSDSSDAGGVLNAYQLTA |
| | | PLESFSKLGKQSGILFYVPAAFTSVIDPTTGFVDLFNSSSITST |
| | | QKKKEFLQRFESIVYSARDGGIFAFTFDYRNFSKIATDHRN |
| | | MWTVYTHGERIRYVRDEKCYKTTDPTKRIKEALSGIEYDD |
| | | GSDIRDKITQSGDNNLINTVYHSFMDTIKMRNKDGRIDYIIS |
| | | PVKNRNGEFFRSDYKHRDFPVDADANGAYHIALKGELLMR |
| | | MIGKTYDSNSDKMPKLEHKDWFEFMQTRGDQ |
| 4 | PAL004 | MCVSRLPWFNITLTGKLNRQRLNQMCVSRLPWFCTPKGQL |
| | (Cas12b) | AATPKTVVAQQENAMLAIIRDVHEAAPADLKTVAQRLEPG |
| | | YFVTQFPKQQMTGDEARAEAERLFAACQKKFKELAEYED |
| | | GYRQCLDALGPNLSLPRLGRKPKGAYPYAVVFKLMPTNAT |
| | | WECFKRVTASLYKRAQKGVVSPVSADSIADVRINDEPLFE |
| | | YFTNLALVRPPGNKDRAVWFEFDLAAFIEAIKSPHQFFQDTI |
| | | KREQAVAQIKAKLDAMDGQGRAASGEEDALPGFEGDDRIT |
| | | LLRELVTDTLGYLAEADASTSPGGKIEYSIQERTVRGFAEV |
| | | KRRWRDLVEKGKATEDALLKVLAEEQTEHRDDFGSATLY |
| | | RELAKPKFQPIWRDPGTQPWHADDPLRAWLEYRELGRELE |
| | | DKQRPIRFTPVHPVHSPRFFIFPKKKGGGRFGTVHEPGQLRV |
| | | MAGIVAQTQHGWEPVPVRITYAAPRLRRDQLRDDVETDLE |
| | | SRPWLQPMMQALGLPEPDTADFSNCRVTLQPSAPDDIQLTF |
| | | PVDVSADKLTTAIGKAARWAKQFNLFPDGDNFYNASLRW |
| | | PHEKKPSKPPVPWHEALDNFSVLAADLGQRCAGAFARLEV |
| | | RANDDFAGKPSRFIGETPGKKWRAALVAAGMLRLPGEEQT |
| | | VWRPGATGPNFHTELSGSRGRMARPHEADDTADLLRAFDC |
| | | PEESLMPADWRTSLSFPEQNDKLLVAARRYQSRLARLHRW |
| | | CWFLTDEKKRQTALDEIREAEDMPAADDPQLTDKLRALLL |
| | | QKQAALPGLLVRLANRILPLRGRSWQWETHPDKADCHLLT |
| | | QTGPALPDVWIRGQRGLSMQRIEQIEELRRRFQSLNQMQRR |
| | | EIGGKPPIRRDDSIPDCCPDLLDKLDQIKEQRANQAAHMILA |
| | | EALGLRLAPPPADKRQLRASRDVHGQYVKSREPVDFIVIED |
| | | LSRYRSSQGRAPRENSRLMKWCHRAVRDKLRELCEPFGIP |
| | | VVETPAAYSSRFCSRSGVAGFRAVEVGPGFDREFPWMMLK |
| | | DREDEGEPVRQLILQVATLNQGRDGKPPRTLLAPLAGGPIF |
| | | VPIVDKLNGADIQPALAQADINAAINLGLRAIADPRLWSIHP |
| | | RCRTQRQGDQMLTREKRKFGETGQPLAVHRADGVKPDDT |
| | | RNPNFFADISGSLPAWESATLDGQHLLSGRCLRSEIKKRQW |
| | | QRCAEINDRRMNRWMKGE |
| 5 | PAL005 | MTELQTQRAYTLRLKGIDEKDQSWRDALWKTHEAVNKGA |
| | (Cas12b) | KVFGDWLLTLRGGLDHTLADAEIPGEKGKPDRAPTQEERK |
| | | HRRILLALSWLSVESERGAPEEFIVATGKEPAATRNDKVIA |
| | | ALKDILRGRNLTEEKISEWTEVCTPSLSAAIREDAVWVNRS |
| | | RAFDEAVKRIGSSLTREEVWDMLECFFGSRNAYLAPVKISE |
| | | DESSDGEQEEKAKDLVQKAGQWLSSRFGTGEGADFAKMA |
| | | AVYAKIAAWAGNAQAGTTGNEVINNLATALREFTPKSNDL |
| | | KGVLDLISGPGYKSATRNLLKQIANTKTVTREDISKLQETA |
| | | GEDSEECATKTGSKGKRAYADAILKDVESVCGFTYRIDKD |
| | | GQPVSVADYSKYDEDYKWGSSRHKEFAVMLDHAARRVSL |
| | | AHTWIKRAEAERRKFEEDSKKIMQVPQAAKDWLDAYCAQ |
| | | RSEASGALEPYRIRKRAIQGWKEIIASWNKPDCKTAEDRIA |
| | | AARQLQDDPEIEKFGDIQLFEALAEDDAQCVWKKEDGTLD |
| | | PEILINYTLASEAMFKKQHFKVPSYRHPDAFLYPVFCDFGN |
| | | SRWELDFSIREAATKLKEIEAKIEKQRQEVHKVQQALEKCE |
| | | NDEKRPKMEERLKEAQKKLQESQNYGEYLHSNNKITMVLF |
| | | DGTFVKKHIFAWQSKRLTKDLALYQEPSADPKNVVSRADR |
| | | LGRAVASVGINDAVKVAGLFEQENWNGRLQAPRQQLEAIA |
| | | QYVEKHGWDNKAEKMRASIKWFITFSAKLQSKGPWNEFA |
| | | RKHGLKEDPHYWPHAEKNENRTAHSRLILSRLPGLRVLSV |
| | | DLGHRYAAACAVWEALGSEAFKKDIEGKRIIRGDTDENAL |
| | | YCHTEHEANGKKHITIYRRIGADTLPDGAHHPAPWARLDR |
| | | QFLIKLQGEDEQAREASNEEIWKVHQLENTLGRRTPLIDRLI |
| | | AGGWGYTEKQKARLEVLTNLGWCPTNKTDNQEEGDEEET |
| | | AILSKPSLLVDDLMESAVRTLRLALKRHGDRARIAHYLITD |
| | | EKTKPGGVKEKLDKNGRVELLLDALGLWHDLFSSPGWHD |
| | | EKAKQLWNAYIAGLLPEGELQQAKSVTTSAALGGQQKKE |
| | | KKEKLRAVAEALYLNSDLCHSLNEVWRKRWEEDDKQWRI |
| | | YIRWFKDWIMPRGANAKSPAIRHVGGLSLTRLATLTEFRRK |
| | | VQVGFFTRLHPDGTKTETREDFGQKTLDTLEHLREQRVKQ |
| | | LASRIVEAALGIGSEDKRHWDGKKRPRQRIADPRFVPCHAV |
| | | VIENLTHYRPEETRTRRENRQIMEWASSKVKKYLSEICQLH |
| | | GLHLREVSAAYTSHQDSRTGAPGIRCQDVSLIEFMKSPFWR |
| | | KQVAQAEKKQKEGKGDAVERYLCELNQKWKGASEEEWR |
| | | KAGFVRIPLRGGEIFVSAAGHDSPAAKGIHADLNAAANIGL |
| | | RALLDPDWSGKWWYVPCNSSTMCPARDKVTGSAAVNPG |
| | | QPLQVSAQLESDDAAKDTKKRKKKGDGKSKEIINLWRDISS |
| | | YPLEDTRGGTWSNKTVYWNRVQSNVVHILQNQMKG |
| 6 | PAL006 | MPETTQRAYTLRLQGHDPKDASWREALWKTHEAVNRGA |
| | (Cas12b) | KAFGDWLLTLRGGLDHSLADEGAPGQTPTEEQRKQRRILL |
| | | ALSWLSVESENGAPQEYIVPHDRDNESGARQNWKTREALR |
| | | EILKNRGCRDDEIESWCHDCEPSLTSAIRKDA VWVNRSKAF |
| | | DNAVQSIPNFSREEIWDLLGCFFVSSQAYLAPLESPKDDKPD |
| | | ASKKDSSKDLIQSAGQWLSRRFGRGKGLNFARLAETYEAIA |
| | | RWASVANPGDTNDLIADLAKTLNAETPELDGILKVVSGPG |
| | | HKSKTRNLLRSLSAVNHITKDTLQRLKDTANEDAKKAKLK |
| | | KGEKGHRAYAYKVLEAVEDACGFTYLQEGDRAKHCEFAV |
| | | MLDHAARRVSSLHTWIKRAEAERRRFEIDTKKKDQLPPSV |
| | | KEWLDTYCQKRSKETGAVEPYRIRRGAIEGWKEIVEAWSK |
| | | AGTTTAEDRKHEARRLPDNPHIDKSGDIKLFEDLALEDALP |
| | | VWHANGDPNNPPDPQLLIDYVEGSEAEFKKRAFKVPTYCH |
| | | PDPLVHPVFCDYGCSRWNVSFAIQPVKKQKLSSEEKLPAKG |
| | | LLLDLLHGTAIRPVALRWQSKRFARDLALNTTDSSDKPNEV |
| | | TRADRFGCALAKCPSSQKIRIRGLFEEKYWNGRLQAPRPEL |
| | | TALAKRVAKYGWDKKARKLRNSLNWFITFSANLRPSGPW |
| | | EEYTKYAEKAFSSNASAKPSVSRGGFWVVHASPNKRGKM |
| | | AQLRLCRLPELRVLSVDLGHRYAAACAVWETLSKSAFEQE |
| | | IHERKILRGGTGPNDLFCHTQHDTNGQSKVTIYRRIGADTLP |
| | | NGTPHPAPWARLDRQFLIKLPGEEREARKASPTELANVEKL |
| | | EKELGLKTSENRVKRIDDLMSDTLRTVRQALRRHSLRARIA |
| | | FNLATLRDQSDGDEESQSKQKRDTRWNNTVKIWHSLLESN |
| | | EWTDDWAKALWDELGPLSDPQKADDAEWLKLAAEKFYT |
| | | RWQEDEQTWRERLRWLRRWILPRGSQAASQKGSIRHVGG |
| | | LSLTRLATIKTLYQVLKAYHMRLKPDNSRKNIPAEGDEALQ |
| | | NFGQKILDDLEHMREQRVKQLASRIVEAALGLGRMKQVTI |
| | | GKDPKRPREPVDQSCHAVVIENLTHYRPEKRQTRRENRQL |
| | | MDWSAAKVKKYLKECCQLHGLHLVEVSASYTSRQDSRTG |
| | | APGIRCQEVPLTDFLKKNFWREQVKQAKQRLSEGKANARD |
| | | RYLCQLNERWGNAPAPVTQTAIRLRIPLNGGELFVSADQNS |
| | | PASKGIQADLNAAANIGLRAITDPDWPGAWWYVPCEANTF |
| | | KPVKDKVAGSAAIDSNVSLKKDSPNSEKPASDRKSRTSKS |
| | | MINLWCDTSSKSLSEKDQWQESAPYWEDVAARTINILQAS |
| | | LACSTTNSQ |
| 7 | PAL007 | MKSLAQFQNLYALQKTLRFELKPEGHTRETFNRWLEEIEKE |
| | (Cas12a) | QASENENIVYQDLLRAKKYEKIKIILDEYHKDFIEQALAYA |
| | | NLTELEKYEELYRKSNRTSEEEEEFENTKESLRKQIANIFIKN |
| | | PNKTVQERWKFLFSKKLIQNELIVWVKGNYELLSEKLKNEF |
| | | PDESSIISTIEDFKYFTTYFRNYHENRKNLYSNEDKESTIAHR |
| | | LIHENLPKFIDNIAIYQKAKAVLNINEVEKELGLPEDTLDKIF |
| | | SLDFFSKALTQKGIDQYNYFLGGKTENEVKKIKGLNEFINL |
| | | YNQQQQDKNQRLPFLKVLYKLPLFERTSTSFRFEPIENDRD |
| | | LIERIGKFYYNDLKQYRDDSQGDTTDILSGINTLLRHVHDY |
| | | RDGLYVNGGITLTQISQKIFGSWSYINNALAYFYDTYIDASG |
| | | VDHQGERKPKKQKQIQEKTKWLKQKQFPVILVEKALSEYK |
| | | SIETNEDLKTRISDTTLCDFFKRCGNDDNGQDLFDRIEARLR |
| | | EKNEEGYSLEDLLKKEFTTERKLMQDKTKTLLIKNFLDVIQ |
| | | GDKDDITAGLLHFVKCLIPRTEISEKNELFYSGMEKYLNILS |
| | | EVTPLYNKARNYLTQKPYSIEKVKLNFENSTLLDGWDENE |
| | | ESDNSCVLLRKRGYYYLGIMNKKHNMIFDRKIYPKATEGE |
| | | AYYEKMIYKLLPGAYKMLPKVFFSEKNIDYFKPSEEILRIRN |
| | | TASYSKNGQPQEGYQKASFSIEDCRKYIDFFKKCIANHWD |
| | | WQKFNFNFSPTEYYQSIDEFYREIERQGYKIDFVKIPESYINQ |
| | | LIKENKLYLFKIYNKDFSEKKKSKGKDNLHTLYWKMLFDE |
| | | KNLKDVVLKLNGEAEVFFRQKSILYNEEIWNKGHHYSELK |
| | | DRFSYPIISNKRYAEDKFFLHVPITLNFKADGINNVNNMVN |
| | | EFIKDNRDIHIIGIDRGERHLLYVSVINQKGDIVEQCSLNEIV |
| | | TEYNGKIFKKNYHEELDNLEKERDRARKDWQTIANIKELK |
| | | EGYLSHVIHKISKLILKYNAIVVMEDLNSGFKRGRQKVEKQ |
| | | VYQNFEKQLIEKLNYLVLKESNVDEPGGVLRAYQLANKFE |
| | | TFKKLGKQSGIIFYVPAAYTSAIDPVTGYIQYLYPLKQADSV |
| | | EKARKFYSQFKRISYNPHKQWFEFSFDYNDFNIIYHGKSSW |
| | | TICTTNTERYMWNRLLNNGHGGEELVYVTNELELLFGEYN |
| | | IIYGDGKDIKQQITDVQDIDVDRTAKQFYKRINELLNLTLKL |
| | | RHNNGKKGADEEDYILSPVEPYFDSRFESRKPSMQQTLPIN |
| | | ADANGAFNIARKGLLLLERLNQLGVEEFEKTKKSNNKKTQ |
| | | WLPHELWVEYAQNHTRK |
| 8 | PAL008 | MAYQNGKEQPTVTNQRAYTLRLSGTNDQDSIWRNRLWHT |
| | (Cas12b) | HEAVNKGAKTFGDWLLTMRGGLCHTLAEADVPGKGNKP |
| | | ARHPTPQEIRSRRVVLALSWLSVESQHGAPERHLVSHDLDI |
| | | ATGERKNWKTVEALREILHGRCLCKELIDEWANDCRDSLS |
| | | ATIREDAVWVNRSKAFDLAAKKIGASLTREELWDFLQPFF |
| | | ANKHGYLQMDTVAGVTNGDSETDAEEAKEDSSEEKAKDL |
| | | SQKAGQWLSSRFGTGTGADFSRFSKVYEVLAARCGSVAVG |
| | | VSGVEAIRILAGTLADFSPGSNDIEGMLGLMSGPGYKSATR |
| | | NILQKINTLQTVSQQDLDRLREASEKDALQSKQKVGGKGS |
| | | RPYANAILQDVEAACGICYAGTGESPARHWQYAVILDHAA |
| | | RRVSMAHSWIKRAEEQRSKFEIEKDKLDHVPKDALAWLDA |
| | | FCARRSSESGASDAYRIRRSAVDGWKQVVAAWAALPPKPE |
| | | NQGSELLSDAESARIQAARELQDTVEKFGDIQLFEALSLTG |
| | | AKCVWQPDGRPDAQPLLDYVAGTDAISKKQRFKVPAYRH |
| | | PDALLHPVFCDFGNSRWNINYAIHRAPEKLTPAQQLLEKKK |
| | | AEIDKAELTLAKAGDAAKQANISEKINGLRAAFIQQQEKVA |
| | | WLNSRHAMTMSLWDGTHIEDTPLIWQSKRFGSDIGQPVEA |
| | | QPLPVSRADRFGRAVALAQDNVPVIPSGLFDLSDWNGRLQ |
| | | APRRQLEAIAAIRDSAKLSVNEKQQLVAKRIQSIRWLLTFSA |
| | | KLQSHGPFIAYAAQHGFDWRYGAHGPENKSRQGLAKLILC |
| | | RLPGLRILSVDLGHRYAAACAVWETLNAGQIQKACLDAGK |
| | | EAPGPCTLYLHLKQIANGKEKKTIFRRIAADTLPDGSPHPAP |
| | | WARLDRQFLIKLQGEDRDARLATSEEIAAVEQMENELGVV |
| | | RQLKRKGRELLVDELMSDALRTLRLGLRRHGVRARIAFNL |
| | | TANKRIRPGGKEEVLDQEGRVLLLTETLLAWYELYTAERW |
| | | TDEPARELWNRHIQPLLGATILQNTVNQEDTPSAAKRRKLR |
| | | EETSGKLKHVAEEIAKNDSLCRQLHVLWSAQWQTEDVIWR |
| | | TRLRMMRRWLLPRGVKRNAQLRISIRDVGGLSLTRIASFKS |
| | | LYQVQKAYQMRPHPEDPRLNIPERGDSRLENFGQRVLDAM |
| | | ERMRENRVKQLASRIAEAALGIGGETGISSKDGSQKKRPTE |
| | | RSSDPRFAPCHAVVIEDLTHYRPDETQTRRENRQLMSWSSS |
| | | KVKKYLGEACELNGLYLREVSPAYTSRQDSRTGAPGLRCN |
| | | DVTVVEFNNSPFWRKQVGAAEKNQKEGNKGDARERYLLS |
| | | IEEGIRGAANDRDIFRIPVKGGEIFVSACITDGGNNAKKNAP |
| | | PGLQADLNAAANIGLRAIFDPDWEGRWWYIPCDAATLCPD |
| | | AKKFIGCKAVDPTKPLRVVAEEGAISASGIGSKKSGRKKNA |
| | | ATDGTRIVNLWRDPSGAPIHRDVLRSPEWQDYAGYWNEV |
| | | QHRVIRNLKTCYEQTSQQEDPFVSQDADKPF |
| 9 | PAL009 | MKRLAETALADKVKCETNSRPKGERAYANSILHDVESACG |
| | (Cas12b) | FTYRVDKGEQPVPVSDYSHYANDYRWGPANHSEFAVMLD |
| | | HAARRVSLAHTWIKRAEAERRQFEENAKKIDKVPKVARE |
| | | WLDSLCAERSIVLGALEPYRIRRRAVDGWKHVVAAWSKS |
| | | DCKTAQDRITAARLLQEDPEIDKFGDIQLFEALAEDHAVCV |
| | | WQRDGEAGKTSDPQLLIDYALAAEAEFKKRHFKVPAYRHP |
| | | EAFWHPVFCDFGQSRWKICFDVHKNRQSRRQRACANRISR |
| | | KICFDVHKKRQTLRLSLEVWTGSKMLDMPLCWQCKRLAR |
| | | DLALGQDHKKDRSCQVTRADRLGRAVSNVARNQEVQILG |
| | | LFEQEYWNGRLQAPRPQLEALGRYIEKHGWDAKAQKSCR |
| | | AIRWMISFSPRLQPAGPWGKFAEKLQLNPNPKYWPHAEDN |
| | | KDRGSRSKLILCRLPGLRVLSVDLGHRYAAACAVWEAVDA |
| | | EQVKEACQAAGHREPNENDLYLHLKKRTTKQKKGSQGVV |
| | | EETTIYRRIGADTLPDCTPHPAPWARLDRQFLIRLQGEEDEA |
| | | RAASNEEVWAVHKLEAELGRTIPLIDRLLGAGWGQTEKQK |
| | | ARLKALRELGWTPANKCQAFNSTDETELRRPSLAVDELML |
| | | DAVGTLRLALKRHGDRARIARYLITDERTKPGGVKEKLDE |
| | | NGRIELLQDALIIWHGLFSSPRWRDDAAKQLWNEHIAKLV |
| | | GEQNLVEVSEDASGSERRTKQKQNREKLREAAKALVDDV |
| | | ALRQALHDMWKRRWEEEDREWRRRLRWFKDWVLPRREQ |
| | | ARKAYSRPAETGSSSHPKRRARYAAIRRVGGLSLTRLATLT |
| | | EFRRKVQVGFFTRLKPDGTKAEAKEGFGQSTLDALEHLRA |
| | | QRVKQLASRIVEAALGVGRIRRFPGVKNPKRPDTPVDKPCH |
| | | AIVIENLTHYRPEETRTRRENRQLMTWSSSKIKKYLAEACQ |
| | | LYGLHLREVTAAYTSRQDSRTGAPGLRCQDVPVKEFMRSL |
| | | FWRKEVAQAEKKLTAGKGSSYERLLCELNQRWKDNSPGD |
| | | GKRAELLRLPHKGGEIFVSAAPDSPAARGLQADLNAAANIG |
| | | LRALTDPDWPGKWWHVPCNAVTFRPVEDKVKGSAAVKL |
| | | DQSLRQVAHPQSKDPGAKKSKEIVNLWCDISSLPLEHREW |
| | | KLDWEPYPAYWNNVQCRVIRVLQGKV |
| 10 | PAL010 | MANAKVKTTTRSYTLSLNAPSDTTDRSPLWHRIFRTHYAIC |
| | (Cas12b) | CGAREFGKLLLDLRGGLPTSLAQLGEGIAENDRRQTQRGTR |
| | | RILALGWLSVEDLDHARNDPHRVQDTAPGSPLDQDLAEKI |
| | | LRKILITKGIKSEEEQSNWISDCLPALTANIRPDAVWVNRAE |
| | | SFAQWQRGTQPGAQPPTPEEAQQILFSLCGESLVTLTLPEQP |
| | | AAAGQKQPDQETSPDPEEQTDRPPAAPSADDEMDPSNASR |
| | | GIFGDLFGENAEGKRSRSQGKDNFACAVRDFLCANPTPSAD |
| | | AITEFREKQKPREPNPPGPEKYPPEVSTSGAPTAVAKRYRKL |
| | | LVCAGLWPKSADEDGSSRNSAKTKFADPKEPQKTEIQINAL |
| | | DLIDACNQAAPADDSGTSPKAGRVFAPAWASNIAEKVASA |
| | | TQMPANAKSLNEFKRLMFALAARRFSQTQSWTRRNEAER |
| | | HMAAARQDAAVARLREIDPDHKAQDWLRGYEQRRADQS |
| | | GSNGEFRITRRMIGEAEAVFKAWAGTNSAAERELKTVAVQ |
| | | TTAEKFGDAALYSEIARNTAAEAVWRSGSAPEILDQWVKL |
| | | RKAQSDQQRTRVPRFCHPNAFRHPTWCEFGESSKPGVWYA |
| | | WNPKSKPRKPEVGGEGDGTRRLWVLLPDFNSGIGQAVPLR |
| | | WRSKRLSKDLGEALQPSDAPIPRADRVSIAAAGLNLEGANG |
| | | VPARYRPSLPFSENTKGWNARLQANRTALLHLESKWDAEA |
| | | ATWRDGGRSLLALKWFTTFSPELAMSEGPGRAIHPKLGWN |
| | | SEPHSDLNRAQKRGGNAKLILSRLPGLRVLSVDLGHRYAA |
| | | ACAVWETLTTEQMNAACQAKNHTQPAESDMYVHLAHPTE |
| | | RVVKSGRKKGQNLIQTTVYRRIAADTLPDGTPHPAPWGRL |
| | | DRQFLIKLQGEQRPTRAASKNEADLANALFHRLGLRSDAD |
| | | SENKSRAVDKLMARTVRVATLGLKRHARRAKIAYALDPN |
| | | TKAIPGMGGSSAAFTPGDEPHIHLLTDALFDWQSLATDAK |
| | | WDDAHARSLWNHHIATLPGGFHLENPTPRDESAHEPSRQR |
| | | QRSGDDALRATLKPIAEKLSKADRQEVHAAWKKYWGDSD |
| | | GQSAIVPKVLQGQRGPEKTTPSASASGWHGKIRWITDWIM |
| | | GKYLEGCTGHAWKHDVGGLSVSRITTMKSLYQLHKAFAM |
| | | RATPEKPRGAPEKGESNLGAAQGILTAMESMRQQRVKQLA |
| | | SRIAEAALGAGIERRSDNGRELQRPRERVDDPRFAACHAVV |
| | | VEDLTNYRPDEMQTRRENRQLMQWASSKVKKYLSEACQL |
| | | HGLYLRGVPAGYTSRQDSRTGAPGVRCGDIPVEELMAAPR |
| | | WRRQILTAEKTRRENNTGTARDRYILTLDEKYRLLTAEQR |
| | | KKTPPARIPVKGGDLFVSADPDSPAASGIQADLNAAANIGL |
| | | KALIDPDWPGRWWYIPCDATTHKPSPERTRGSAAVDCDVP |
| | | LGPDSTGTPEDRDAKPKKNQRNSKIAGRGQSAIINLWRDPT |
| | | HLPIKENPSAWCESKKYWNQVEHNVVKVIESKGQKLTQTA |
| | | EAATGESASSPPIAPTDVPW |
| 11 | RS9 | MEEKMLKSYDYFTKLYSLQKTLRFELKPIGKTLEHIKNSGII |
| | | ESDETLEEQYAIVKNIIDKLHRKHIDEALSLVDFTKHLDTLK |
| | | TFQELYLKRGKTDKEKEELEKLSADLRKLIVSYLKGNVKE |
| | | KTQHNLNPIKERFEILFGKELFTNEEFFLLAENEKEKKAIQA |
| | | FKGFTTYFKGFQENRKNMYSEEGNSTSIAYRIINENLPLFIE |
| | | NIARFQKVMSTIEKTTIKKLEQNLKTELKKHNLPGIFTIEYF |
| | | NNVLTQEGISRYNTIIGGKTTHEGVKIQGLNEIINLYNQQSK |
| | | DVKLPILKPLHKQILSEEYSTSFKIKAFENDNEVLKAIDTFW |
| | | NEHIEKSIHPVTGNKFNILSKIENLCDQLQKYKDKDLEKLFI |
| | | ERKNLSTVSHQVYGQWNIIRDALRMHLEMNNKNIKEKDID |
| | | KYLDNDAFSWKEIKDSIKIYKEHVEDAKELNENGIIKYFSA |
| | | MSINEEDDEKEYSISLIKNINEKYNNVKSILQEDRTGKSDLH |
| | | QDKEKVGIIKEFLDSLKQLQWFLRLLYVTVPLDEKDYEFYN |
| | | ELEVYYEALLPLNSLYNKVRNYMTRKPYSVEKFKLNFNSP |
| | | TLLDGWDKNKETANLSIILRKNGKYYLGIMNKENNTIFEYY |
| | | PGTKSNDYYEKMIYKLLPGPNKMLPKVFFSKKGLEYYNPP |
| | | KEILNIYEKGEFKKDKSGNFKKESLHTLIDFYKEAIAKNED |
| | | WEVFNFKFKNTKEYEDISQFYRDVEEQGYLITFEKVDANY |
| | | VDKLVKEGKLYLFQIYNKDFSENKKSKGNPNLHTIYWKGL |
| | | YDSENLKNVVYKLNGEAEVFYRKKSIDYPEEIYNHGHHKE |
| | | ELLGKFNYPIIKDRRYTQDKFLFHVPITMNFISKEEKRVNQL |
| | | ACEYLSATKEDVHIIGIDRGERHLLYLSLIDKEGNIKKQLSL |
| | | NTIKNENYDKEIDYRVKLDEKEKKRDEARKNWDVIENIKE |
| | | LKEGYMSQVIHIIAKMMVEEKAILIMEDLNIGFKRGRFKVE |
| | | KQVYQKFEKMLIDKLNYLVFKNKNPLEPGGSLNAYQLTSK |
| | | FDSFKKLGKQSGFIFYVPSAYTSKIDPTTGFYNFIQVDVPNL |
| | | EKGKEFFSKFEKIIYNTKEDYFEFHCKYGKFVSEPKNKDND |
| | | RKTKESLTYYNAIKDTVWVVCSTNHERYKIVRNKAGYYES |
| | | HPVDVTKNLKDIFSQANINYNEGKDIKPIIIESNNAKLLKSIA |
| | | EQLKLILAMRYNNGKHGDDEKDYILSPVKNKQGKFFCTLD |
| | | GNQTLPINADANGAYNIALKGLLLIEKIKKQQGKIKDLYISN |
| | | LEWFMFMMSR |

### Detectably Labeled Nucleic Acid Probe

A detectably labeled nucleic acid probe according to the present disclosure is susceptible to preferred collateral cleavage by a Cas enzyme. In some embodiments, a detectably labeled nucleic acid probe according to the present disclosure is characterized by having a detectable entity (e.g., a reporter dye). In some embodiments, a detectably labeled nucleic acid probe according to the present disclosure is characterized by having a detectable reporter dye. In some embodiments, a detectably labeled nucleic acid probe according to the present disclosure is characterized by having a particular sequence length. In some embodiments, a detectably labeled nucleic acid probe according to the present disclosure is characterized by having a particular type of nucleic acids.

In some embodiments, a nucleic acid probe sequence is a non-target nucleic acid sequence. In some embodiments, a nucleic acid probe sequence comprises a cleavable nucleic acid probe sequence. In some embodiments, a Cas enzyme cleaves the detectably labeled nucleic acid probe comprising a reporter dye with preferential specificity relative to an otherwise comparable detectably labeled nucleic acid probe characterized by having a different reporter dye.

In some embodiments, a reporter dye is attached to the 5′ end of the probe sequence. In some embodiments, a reporter dye is attached to the 3′ end of the probe sequence. In some embodiments, a reporter dye is a fluorescent reporter dye. In some embodiments, a reporter dye is a fluorophore. In some embodiments, a reporter dye emits a reporter dye signal when it is no longer attached to the nucleic acid probe sequence (e.g., because it is separated from a quencher that is also attached to the probe sequence). In some embodiments, a reporter dye is selected from the group consisting of Cy5, FAM, TxRed, YakYel, and HEX. In some embodiments, a reporter dye signal (e.g., florescence) can be determined.

In some embodiments, the present disclosure provides technologies (e.g., compositions and methods) utilizing a plurality of detectably labeled nucleic acid probes (e.g., a first detectably labeled nucleic acid probe and a second detectably labeled nucleic acid probe).

In some embodiments, a detectably labeled nucleic acid probe according to the present disclosure is characterized by having a sequence length of about 4 to about 20 nucleotides, about 5 to about 15 nucleotides, about 7 to about 12 nucleotides. In some embodiments, detectably labeled nucleic acid probe sequence lengths comprise about 7 to about 12 nucleotides. In some embodiments, a detectably labeled nucleic acid probe sequence is at least 7 nucleotides in length, at least 8, at least 9, at least 10, at least 11, at least 12, at least 13, or at least 12 nucleotides in length. In some embodiments, a first and second detectably labeled nucleic acid probe have the same sequence length. In some embodiments, a first and second detectably labeled nucleic acid probe have a different sequence length.

In some embodiments, a first and second detectably labeled nucleic acid probe have the same length and comprise the same nucleic acids. In some embodiments, a first and second detectably labeled nucleic acid probe have the same length and comprise different nucleic acids.

In some embodiments, a first and second detectably labeled nucleic acid probe have different lengths and comprises the same nucleic acids. In some embodiments, a first and second detectably labeled nucleic acid probe have different lengths and comprises the same nucleic acids.

In some embodiments, a detectably labeled nucleic acid probe sequence comprises at least one thymine nucleotide. In some embodiments, both detectably labeled nucleic acid probe sequences comprise at least one thymine nucleotide. In some embodiments, at least one detectably labeled nucleic acid probe sequence comprises about 5 to about 15 thymine nucleotides, about 7 to about 12 thymine nucleotides. In some embodiments, both detectably labeled nucleic acid probe sequences comprise about 5 to about 15 thymine nucleotides, about 7 to about 12 thymine nucleotides. In some embodiments, at least one detectably labeled nucleic acid probe sequence comprises of 95%-100% thymine nucleotides. In some embodiments, both detectably labeled nucleic acid probe sequences comprise of 95%-100% thymine nucleotides. In some embodiments, at least one detectably labeled nucleic acid probe sequence comprises at least one cytosine nucleotide. In some embodiments, both detectably labeled nucleic acid probe sequences comprise at least one cytosine nucleotide. In some embodiments, at least one detectably labeled nucleic acid probe sequence comprises about 5 to about 15 cytosine nucleotides, such as about 7 to about 12 cytosine nucleotides. In some embodiments, both detectably labeled nucleic acid probe sequences comprise about 5 to about 15 cytosine nucleotides, such as about 7 to about 12 cytosine nucleotides. In some embodiments, at least one detectably labeled nucleic acid probe sequence comprises of 95%-100% cytosine nucleotides. In some embodiments, both detectably labeled nucleic acid probe sequences comprise of 95%-100% cytosine nucleotides.

In some embodiments, a detectably labeled nucleic acid probe according to the present disclosure comprises a quencher. In some embodiments, a quencher is selected from the group consisting of 3IAbRQsp, BHQ2, 3IABKFQ, and BHQ1.

In some embodiments, a quencher is capable of suppressing a reporter dye signal (e.g., fluorescence of a reporter dye) as provided herein above when they are both attached to the probe nucleic acid sequence. In some embodiments, a reporter dye and a quencher are attached (e.g., covalently linked) at opposite ends of a probe nucleic acid sequence. When the probe nucleic acid sequence is intact and the reporter dye is in close proximity to the quencher, little to no fluorescence will result because of suppression of the reporter fluorescence due to an energy transfer between the two dyes. Upon recognition of a target nucleic acid sequence, Cas enzyme's collateral activity is activated; leading to cleavage of bystander detectably labeled nucleic acid probes causing separation of the fluorophore and quencher leading to fluorescence (e.g., after cleavage of the probe nucleic acid sequence the reporter dye is released from the probe sequence and the quencher, and is no longer in close proximity to the quencher and it can fluoresce). In some embodiments, a detectably labeled nucleic acid comprises relevant cleavable nucleic acids appropriately configured to a reporter dye so that its cleavage as a result of the activated collateral activity is detectable (e.g., separates a fluorophore from a quencher so that fluorescence becomes detectable, etc.). In some embodiments, an increase in the fluorescence signal results if a Cas enzyme collateral activity is activated and the Cas enzyme demonstrates preferential cleavage of the detectably labeled nucleic acid probe.

In some embodiments, a quencher is capable of suppressing florescence of a reporter dye selected from the group consisting of Cy5, FAM, TxRed, YakYel, and HEX.

In some embodiments, a detectably labeled nucleic acid probe comprises a nucleic acid probe sequence comprising or consisting of a nucleic acid sequence, such as a DNA and/or RNA sequence. In some embodiments, a nucleic acid probe sequence is a double-stranded DNA. In some embodiments, a nucleic acid probe sequence is a single-stranded DNA. In some embodiments, a nucleic acid probe sequence is a single-stranded RNA.

In some embodiments, the nucleic acid probe sequence is selected from the group of sequences listed in Table 2. In some embodiments, the nucleic acid probe sequence is selected from the group consisting of T7, C7, T12, C12, rC5 and IN7.

TABLE 2
Nucleic acid probe sequence

| SEQ ID | | |
|---|---|---|
| NO: | Probe | Probe Sequence |
| 12 | T7 | TTTTTTT |
| 13 | C7 | CCCCCCC |
| 14 | N7 | NNNNNNN |
| 15 | T12 | TTTTTTTTTTTT |
| 16 | C12 | CCCCCCCCCCCC |
| | DNase Alert | DNase Alert |
| | RNase Alert | RNase Alert |
| 17 | rC5 | rCrCrCrCrC |
| 18 | rN7 | rNrNrNrNrN |
| 19 | rC16A4 | rCrCrCrCrCrCrCrCrCrCrCr |
| | | CrCrCrCrCrArArArA |

The following exemplary detectably labeled nucleic acid probes may be useful in methods or compositions as provided herein. In some embodiments, a detectably labeled nucleic acid probe is selected from the group consisting of: T7-Cy5, C7-Cy5, N7-Cy5, T12-Cy5, C12-Cy5, T7-FAM, C7-FAM, N7-FAM, T12-FAM, C12-FAM, T7-TxRed, C7-TxRed, T7-YakYel, DNaseAlert™-HEX, RNaseAlert™-FAM, rC5-FAM, IN7-FAM, and rC16A4-FAM. In some embodiments, a detectably labeled nucleic acid probe is selected from the group consisting of: T7-Cy5, C7-Cy5, T7-TxRed, C7-TxRed, T7-YakYel, DNaseAlert™-HEX, RNaseAlert™-FAM, and rN7-FAM.

### Exemplary Detectably Labeled Nucleic Acid Probes

In some embodiments, a first Cas enzyme cleaves a first detectable labeled nucleic acid probe comprising a first reporter dye by recognizing the reporter dye, the length of the detectable labeled nucleic acid probe, the types of nucleic acids in the detectable labeled nucleic acid probe, or combinations thereof.

In some embodiments, a first and second detectable labeled nucleic acid probe as provided herein comprise the same reporter dye, the same length of the detectable labeled nucleic acid probes, and different types of nucleic acids.

In some embodiments, a first and second detectable labeled nucleic acid probe as provided herein comprise the same reporter dye, different lengths of the detectable labeled nucleic acid probes, and the same type of nucleic acids.

In some embodiments, a first and second detectable labeled nucleic acid probe as provided herein comprise the same reporter dye, different lengths of the detectable labeled nucleic acid probes and different types of nucleic acids.

In some embodiments, a first and second detectable labeled nucleic acid probe as provided herein comprise different reporter dyes, the same length of the detectable labeled nucleic acid probes, and different types of nucleic acids.

In some embodiments, a first and second detectable labeled nucleic acid probe as provided herein comprise different reporter dyes, different lengths of the detectably labeled nucleic acid probes and same type of nucleic acids.

In some embodiments, a first and second detectable labeled nucleic acid probe as provided herein comprise different reporter dyes, different lengths of the detectably labeled nucleic acid probes, and different types of nucleic acids.

### Guide Polynucleotides

In some embodiments, technologies (e.g., methods and compositions) according to the present disclosure further comprises a guide polynucleotide.

In some embodiments, Cas enzymes are activated to cleave nucleic acids (e.g., corresponding detectably labeled nucleic acid probes) when complexed with a guide polynucleotide hybridized with a complementary sequence (herein referred to as “target nucleic acid”). It is well established that guide polynucleotides can be engineered to hybridize with any target nucleic acid sequence of interest. Additionally, it is well established that guide polynucleotides may include natural nucleotides, nucleotide analogs, and/or combinations thereof. All of that established knowledge is relevant to, and may be employed in the practice of, the present disclosure.

In some embodiments, a guide polynucleotide comprises a guide nucleic acid sequence with sufficient complementarity to a target nucleic acid that the guide polynucleotide is capable of hybridizing to the target nucleic acid. In some embodiments, a guide polynucleotide and a Cas enzyme is capable of forming a complex. In some embodiments, a guide polynucleotide is capable of forming a complex with a Cas enzyme and directing the complex to bind to a target nucleic acid.

In some embodiments, a guide polynucleotide comprises a guide nucleic acid sequence that is complementary to a target nucleic acid sequence. In some embodiments, a guide polynucleotide sequence is 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more complementary to a target nucleic acid sequence. In some embodiments, a guide sequence is 5, 6, 7, 8, 9, 10, 11, 12, 13, 14, 15, 16, 17, 18, 19, 20, 21, 22, 23, 24, 25, or more nucleotides in length.

In some embodiments, technologies of the present disclosure utilize a plurality of guide polynucleotides. In some embodiments, a plurality of guide polynucleotides comprises 2, 3, 4, 5, 6, 7, 8, 9, 10, 20, 30, 40, 50, 60, 70, 80, 90, 100, 250, 500, 750, 1,000 or more guides. In some embodiments, two or more guides comprise the same guide sequence. In some embodiments, two or more guides comprise different guide sequences. In some embodiments, two or more guide sequences hybridize to different target sites of the same target nucleic acid. In some embodiments, two or more guide sequences hybridize to different target nucleic acid sequences.

In some embodiments, the capability of a guide polynucleotide to direct sequence-specific binding of Cas enzymes to a target nucleic acid may be assessed by any suitable assay. For example, the Cas enzymes and guide polynucleotides disclosed herein are sufficient to form a complex, including the guide nucleic acid sequence to be tested, may be provided to a cell having the corresponding (e.g., complementary) target nucleic acid sequence, such as by transfection with vectors encoding Cas enzymes and/or guide polynucleotides, followed by a characterization of preferential cleavage within the target nucleic acid sequence. In some embodiments, cleavage of a target nucleic acid may be evaluated, for example, in a test tube by providing a target nucleic acid, components of CRISPR-Cas technologies, including the guides to be tested and a control guides comprising guide sequences different from the test guide sequence, and comparing binding or rate of cleavage at the target nucleic acid between the test and control guide sequence reactions. Other assays are possible, and will occur to those skilled in the art. In some embodiments, a guide nucleic acid sequence may be selected to hybridize any target nucleic acid sequence. In some embodiments, a target nucleic acid sequence is a nucleic acid sequence within a genome of a cell. In some embodiments, a target nucleic acid sequence is a nucleic acid sequence in a sample.

### Samples

In some embodiments, technologies of the present disclosure are contacted with a sample. In some embodiments, a sample is or comprises a biological sample. A biological sample typically refers to a sample obtain or derived from a biological source, for example, including a tissue, organism, or cell culture) of interest, as provided herein. In some embodiments, a source of interest comprises an organism, such as an animal or human. In some embodiments, a sample is obtained or derived from an organism (e.g., a mammalian organism, for example, including a human). In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

In some embodiments, a source of interest comprises a virus or microbe. In some embodiments, a sample is obtained or derived from virus or microbe. In some embodiments, a viral or microbial sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, a sample comprises a target nucleic acid.

### Target Nucleic Acid

Those of ordinary skill in the art will readily appreciate that technologies of the present disclosure are broadly applicable to achieve detection of a wide range of target nucleic acids. In some embodiments, target nucleic acids include, for example, nucleic acids from an infectious agent (e.g., a virus, microbe, parasite, etc.), nucleic acids indicative of a particular physiological state or condition (e.g., presence or state of a disease, disorder or condition such as, for example, cancer or an inflammatory or metabolic disease, disorder or condition, etc.), prenatal nucleic acids, etc.

In some embodiments, a useful target nucleic acid in accordance with the present disclosure is not limited to a particular length; in some embodiments, a target nucleic acid is a nucleotide of any length (oligonucleotides or polynucleotides) comprising a sequence to which a guide sequence hybridizes. In some embodiments, a target nucleic acid comprises a three dimension structure. In some embodiments, a target nucleic acid sequence comprises coding and/or non-coding regions. In some embodiments, a target nucleic acid sequence comprises exons, introns, mRNA, tRNA, IRNA, siRNA, shRNA, miRNA, ribozymes, cDNA, plasmids, vectors, exogenous nucleotide sequences, and/or endogenous nucleotide sequences. In some embodiments, a target nucleic acid sequence comprises modified nucleotides, for example, including methylated nucleotides or nucleotide analogs. In some embodiments, a target nucleic acid sequence may be interspersed with non-nucleic acid components. In some embodiments, a target nucleic acid is a single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. In some embodiments, a target nucleic acid is located in the nucleus or cytoplasm of a cell. In some embodiments, a target nucleic acid is ex vivo. In some embodiments, a target nucleic acid is present in an in vitro system. In some embodiments, a target nucleic acid is present in a sample, e.g., in a biological sample or in an environmental sample.

In some embodiments, a target nucleic acid is recognized by CRISPR-Cas technologies (e.g., a guide polynucleotide) and binds a Cas enzyme as provided herein. In some embodiments, a target nucleic acid sequence comprises a specific, recognizable, protospacer adjacent motif (PAM).

In some embodiments, provided technologies are particularly useful or applicable for detection of low-abundance (e.g., less than about 10 fM, or about 1 fM, or about 100 aM) nucleic acids. In some embodiments, provided technologies are particularly useful or applicable for detection of less than about 100 aM, or about 10 aM or about 1 aM nucleic acids.

### Methods of Detection

In some embodiments, the present disclosure provides methods of detection a target nucleic acid in a sample comprising contacting the sample with a composition as provided herein and a first and a second guide polynucleotide.

In some embodiments, detection assays (e.g., multiplex assays) that utilize compositions according to the present disclosure involve contacting the Cas enzymes having collateral cleavage activity and demonstrating preferential cleavage specificity for their corresponding detectably labeled nucleic acid probe and guide polynucleotides complementary to a target nucleic acid sequence of interest with a sample that may contain the target nucleic acid. In some embodiments, upon recognition of the target nucleic acid sequence, the Cas enzyme's collateral activity is activated, so that it cleaves its corresponding detectably labeled nucleic acid probe comprising a reporter dye. In some embodiments, the reporter dye is appropriately configured to the cleavable nucleic acid probe sequence so that cleavage of the nucleic acid probe sequence as a result of the activated collateral activity is detectable (e.g., separates a fluorophore from a quencher so that fluorescence becomes detectable, etc.).

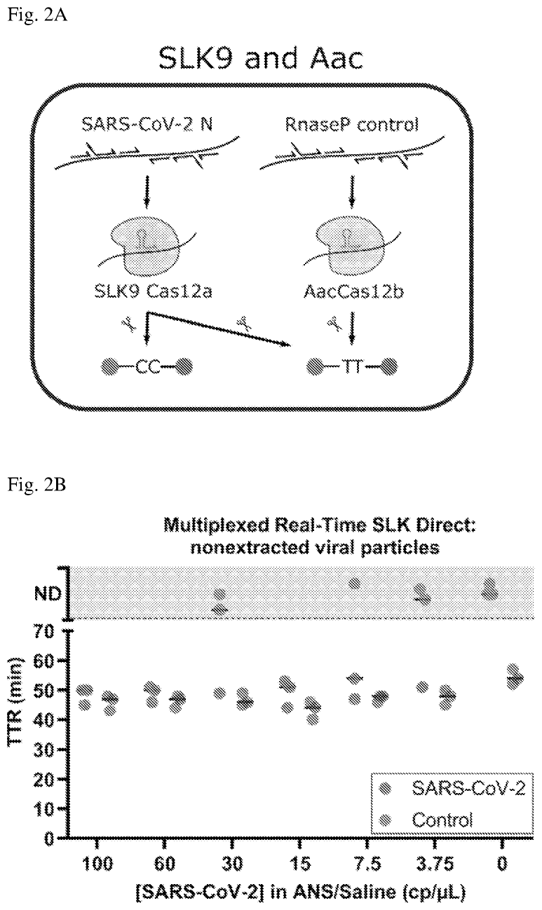

In some embodiments, Cas enzymes as provided herein are particular useful in detection of multiple target nucleic acids in a single system (e.g., a vessel). In some embodiments, technologies provided herein utilize a plurality of Cas enzymes (e.g., a first and a second Cas enzyme) and a plurality of detectably labeled nucleic acid probes having different reporter dyes (e.g., a first and a second detectably labeled nucleic acid probe having a first and a second reporter dye that differ). In some such embodiments, the first and second Cas enzyme and their corresponding first and second detectably labeled nucleic acid probes are mutually orthogonal in the same system (e.g., vessel). In some embodiments, technologies provided herein further utilize a plurality of guide polynucleotides (e.g., a first and a second guide polynucleotide). Without wishing to be bound by any one theory, use of a plurality of detectably labeled nucleic acid probes having different reporter dyes enable detection of a plurality of target nucleic acids (e.g., measuring a plurality of different reporter dye signals). In some embodiments, technologies as provided herein utilize a plurality of detectably labeled nucleic acid probes having different reporter dyes for multiplexing. See FIGS. 2A-B and 3A-B.

In some embodiments, a first guide polynucleotide and a second guide polynucleotide hybridize to one or more target nucleic acids. In some such embodiments, a first Cas enzyme is capable of forming a complex with a first guide polynucleotide hereby activating its collateral cleavage activity and cleave its corresponding first detectably labeled nucleic acid probe to generate a detectably reporter dye signal. In some such embodiments, a second Cas enzyme is capable of forming a complex with a second guide polynucleotide hereby activating its collateral cleavage activity and cleave its corresponding second detectably labeled nucleic acid probe to generate a detectably reporter dye signal. As the reporter dyes are different and the Cas enzymes demonstrate preferential cleavage specificity for their corresponding first or second detectably labeled nucleic acid probe two different signals can be detected when both guide polynucleotides hybridize to a target sequence or a single signal can be detected if only one of the guide polynucleotides hybridizes to a target sequence.

In some embodiments, the present disclosure provides robust multiplexed CRISPR-based detecting using two Cas enzymes (e.g., a first and a second Cas enzyme) to simultaneous detect two different target nucleotide sequences (e.g., diagnose a particular infection and pinpoint the causative variant of concern all while maintaining high levels of accuracy upon detection of multiple gene targets or diagnose a particular infection and a control).

In some embodiments, methods of detecting a target nucleic acid in a sample comprise contacting a sample with a composition according to the present disclosure.

In some embodiments, methods of detecting a target nucleic acid in a sample comprising contacting a sample with a composition comprising (a) a first Cas enzyme having collateral cleavage activity and a first detectably labeled nucleic acid probe comprising a first reporter dye; (b) a second Cas enzyme having collateral cleavage activity and a second detectably labeled nucleic acid probe comprising a second reporter dye, wherein the first and second reporter dye are different; and the first and second Cas enzyme are different and wherein both the first and the second Cas enzyme demonstrates preferential cleavage specificity for its corresponding detectably labeled nucleic acid probe comparable to its non-corresponding detectably labeled nucleic acid probe, and (c) a first and a second guide polynucleotide; wherein hybridization of the first guide polynucleotide and first Cas enzyme or second guide polynucleotide and second Cas enzyme with a target nucleic acid activates the collateral activity and cleavage of a corresponding detectably labeled nucleic acid probe wherein cleavage of a corresponding detectably labeled nucleic acid probe indicates presence of the target nucleic acid.

In some embodiments, methods of detecting a target nucleic acid in a sample further comprises a step of incubating the sample with a composition as provided herein. In some embodiments, the step of incubating is performed for a period of time within a range of 10 seconds to 90 minutes, such as 30 seconds to 40 minutes, such as 5 minutes to 30 minutes, such as 10 minutes to 30 minutes.

In some embodiments, methods of detecting a target nucleic acid in a sample further comprise a step of determining cleavage of a first or a second detectably labeled nucleic acid probe. In some embodiments, methods of detecting a target nucleic acid in a sample further comprise a step of determining cleavage of a first and a second detectably labeled nucleic acid probe. In some embodiments, determining cleavage of the first or second detectably labeled nucleic acid probes comprises detecting a reporter dye signal (e.g., a fluorescence signal) as provided herein above.

To allow for multiplexing, wherein two different reporter signals indicate the presence of two different target nucleic acids, two different reporter dyes are normally use. Thus, technologies using Cas enzymes recognizing the nucleic acid probe sequence and not the reporter dye requires more design optimization compared to using Cas enzymes of the present disclosure that recognize the reporter dye. As Cas enzymes according to the present disclosure recognize the reporter dye

### Kits

In some embodiments, the present disclosure provides kits containing any one or more of the elements disclosed in the above compositions and methods. In some embodiments, a kit comprises one or more of the components of the technologies as provided herein, such as Cas enzymes, detectably labeled nucleic acid probes, guide polynucleotides, vector, and/or vector system (e.g., DNA or RNA) encoding or providing the same.

In some embodiments, a kit comprises a composition comprising a first Cas enzyme having collateral cleavage activity and a first detectably labeled nucleic acid probe comprising a first reporter dye; and a second Cas enzyme having collateral cleavage activity and a second detectably labeled nucleic acid probe comprising a second reporter dye, wherein the first and second reporter dye are different, and the first and second Cas enzyme are different; and wherein both the first and second Cas enzyme demonstrate preferential cleavage specificity for its corresponding first or second detectably labeled nucleic acid probe compared to its non-corresponding detectably labeled nucleic acid probe. In some such embodiments, a kit further comprises a first guide polynucleotide and a second guide polynucleotide.

In some embodiments, a kit further comprises instructions in one or more languages for using the kit. In some such embodiments, the instructions are to a specific application and/or method provided herein. Elements may be provided individually or in combinations. Kits may be provided in any suitable container. In some embodiments, a suitable container is, for example, a vial, a bottle, or a tube.

In some embodiments, a kit comprises one or more reagents for use in a process utilizing one or more of the elements provided herein. Reagents may be provided in any suitable container. For example, in some embodiments, a kit provides one or more reaction or storage buffers. Reagents may be provided in a form that is usable in a particular assay, or in a form that requires addition of one or more other components before use (e.g., in concentrate or lyophilized form). In some embodiments, a buffer is not limited to a particular buffer; in some embodiments, a buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In some embodiments, the kit comprises one or more of the vectors and/or one or more of the polynucleotides provided herein. The kit may advantageously allow to provide all elements of the systems of the disclosure.

### Exemplification

### Example 1: Exemplary Materials and Methods

The present example demonstrates materials and methods used in Example 2.

### Reporter Assay

The final concentration of components in the Real-time SHERLOCK reaction mix are (20 μL final volume): 1x LAMP primer mix, 1x WarmStart® LAMP reaction mix, either 292 nM RS9 enzyme or 250 nM PAL5 enzyme, 112.5 nM guide RNA (for RS9) and 250 nM guide RNA for PAL enzymes. Different detectably labeled nucleic acid probes were used; labeled DNA or RNA was used at 125 nM concentration and both DNase and Rnase Alert were used at 400 nM. Target concentration for SARS-COV-2 was 1000 cp/reaction and for human RNA the concentration used was 40 ng/reaction. All reagents were mixed and the increase in fluorescence was measured every 1 minute for 90 minutes using an Applied Biosystems QS5 qPCR instrument set to 60° C.

TABLE 3
Detectably labeled nucleic acid probe
(all from Integrated DNA Technologies)

| Reporter sequence | Fluorophore |
|---|---|
| TTTTTTT (SEQ ID NO: 12) | FAM, TxRed, Cy5, |
| | Yak Yel |
| CCCCCCC (SEQ ID NO: 13) | FAM, TxRed, Cy5 |
| NNNNNNN (SEQ ID NO: 14) | FAM, Cy5 |
| TTTTTTTTTTTT (SEQ ID NO: 15) | FAM, Cy5 |
| CCCCCCCCCCCC (SEQ ID NO: 16) | FAM, Cy5 |
| Dnase Alert | HEX |
| Rnase Alert | FAM |
| rCrCrCrCrC (SEQ ID NO: 17) | FAM |
| rNrNrNrNrN (SEQ ID NO: 18) | FAM |
| rCrCrCrCrCrCrCrCrCrCrCrCr | FAM |
| CrCrCrCrArArArA | |
| (SEQ ID NO: 19) | |

### Example 2: Exemplary Label-Discrimination

The present example demonstrates use of Cas enzymes in combination with particular detectably labeled nucleic acid probes.

Real-time SHERLOCK reactions (as described in Example 1) were conducted using various combinations of Cas enzymes and detectably labeled nucleic acid probes. The results showed that PAL enzymes (e.g., PAL5) cleave only C7-FAM, but not C7 with other reporter dyes (e.g., TxRed, Cy5) (FIGS. 1A-C and Table 4).

TABLE 4

| Enzyme | Guide | Reporter | NTC | Target |
|---|---|---|---|---|
| PAL5 | O | C7-FAM | 0/3 | 3/3 |
| PAL5 | N | C7-FAM | 1/3 | 3/3 |
| RS9 | RP | C7-FAM | 0/3 | 3/3 |
| PAL5 | O | C7-TxRed | 0/3 | 0/3 |
| PAL5 | N | C7-TxRed | 0/3 | 0/3 |
| RS9 | RP | C7-TxRed | 0/3 | 3/3 |
| PAL5 | O | C7-Cy5 | 0/3 | 3/3 |
| PAL5 | N | C7-Cy5 | 0/3 | 3/3 |
| RS9 | RP | C7-Cy5 | 0/3 | 3/3 |

O: SARS-CoV-2 Orf1ab gene.
N: SARS-CoV-2 N-gene; and RP: RnaseP.

Probe specificity of particular Cas enzymes (e.g., PAL enzymes) was evaluated using a variety of probes (e.g., different combinations of sequence length, sequence nucleotides, and dyes). Exemplary probe specificity of various PAL enzymes are shown in Table 5.

TABLE 5

| Probe | | | | | | | |
|---|---|---|---|---|---|---|---|
| Sequences | Dye | Quencher | RS9 | PAL5 | PAL9 | PAL10 | PAL8 |
| T7 | Cy5 | 3IAbRQsp | X | % | % | % | N/A |
| C7 | Cy5 | BHQ2 | X | % | % | % | N/A |
| N7 | Cy5 | BHQ2 | X | X | X | X | N/A |
| T12 | Cy5 | BHQ2 | X | X | X | X | N/A |
| C12 | Cy5 | BHQ2 | X | X | X | X | N/A |
| T7 | FAM | 3IABkFQ | X | X | X | X | N/A |
| C7 | FAM | BHQ1 | X | X | X | X | X |
| N7 | FAM | BHQ1 | X | X | X | X | N/A |
| T12 | FAM | BHQ1 | X | X | X | X | N/A |
| C12 | FAM | BHQ1 | X | X | X | X | N/A |
| T7 | TxRed | BHQ2 | X | % | % | % | N/A |
| C7 | TxRed | BHQ2 | X | % | % | % | N/A |
| N7 | TxRed | BHQ2 | X | X | X | X | X |
| T7 | YakYel | 3IABkFQ | X | X | X | X | N/A |
| DNase | HEX | | X | % | % | % | % |
| Alert | | | | | | | |
| RNase | FAM | | % | X | X | X | X |
| Alert | | | | | | | |
| rC5 (RNA) | FAM | 3IABkFQ | % | % | % | % | N/A |
| rN7 (RNA) | FAM | 3IABkFQ | % | X | % | % | % |
| rC16A4 | FAM | 3IABkFQ | % | % | % | % | N/A |
| (RNA) | | | | | | | |

“X” indicates that the Cas enzyme cleaved the probe.
“%” indicates that the Cas enzyme did not cleave the probe.

Certain Cas enzymes (e.g., RS9, PAL5, PAL9, PAL10 and PAL 8) showed preferential cleavage of certain probes based on dye and/or sequence (e.g., sequence length and/or type of nucleic acids) preference. Certain Cas enzymes cleave N-probes (e.g., detectably labeled nucleic acid probes) with Cy5, but not T7 or C7 with either Cy5 or TxRed (e.g., PAL5, PAL9, PAL10), whereas certain Cas enzymes cleave N-probes with Cy5 and TxRed.

### Example 3: Multiplex Real-Time SHELOCK

This Example demonstrates real-time multiplex detection of two separate target sequences utilizing two different Cas enzymes and two different probes.

### Materials

Real-time SLK was performed with 40 μL of total reaction volume. Final concentrations of components in the real-time SLK reaction mix were 1xN-replicase polyprotein (Orf)-1-RP primer mix, 1xWarmStart LAMP reaction mix (New England BioLabs), 292 nmol/L RS9 (SLK-9) enzyme or PAL5 (SLK5-2), 368 nmol/L Alicyclobacillus acidoterrestris AacCas 12b, and 125 nmol/L poly-C probe. Corresponding guides at 112.5 nmol/L N/O/RP for RS9 (SLK9), 520 nmol/L for Aac and 292 nmol/L for PAL5 (SLK5-2). The following probes were utilized: C7-probes comprising a TxRed dye, T7-probes comprising a FAM dye, and an RNase Alert-probes comprising a FAM dye. A total of 14 μL of extracted RNA or 8 μL of direct sample was added to the SLK reaction mix. Fluorescence was measured every 1 minute for 90 minutes using the QuantStudio 5qPCR instrument set to 60° C.

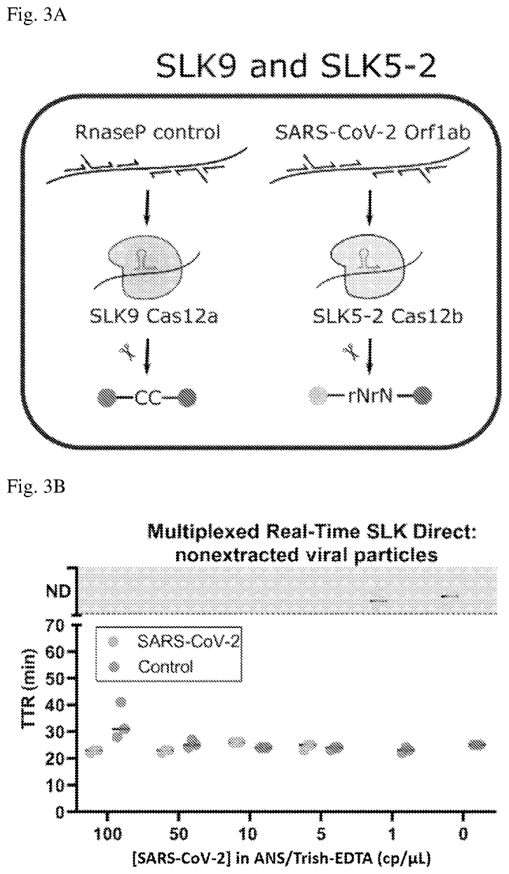

Samples of anterior nasal swab (ANS)/saline (FIGS. 2A-B) and ANS/Tris-EDTA (FIGS. 3A-B) spiked with indicated concentrations of SARS-COV-2 viral particles were treated with Proteinase K, RNAsecure, and heat and directly added to a real-time SLK reaction.

### Methods

The real-time, multiplexed detection system contains, in a single reaction, RT-LAMP components for duplexed LAMP of the SARS-COV-2N gene and the human RP control, RS9 (SLK9) enzyme with Crispr RNA targeting the N gene, AacCas12b enzyme with Crispr RNA targeting the human RP gene, a carboxyfluoresce in-black hole quencher 1 (FAM-BHQ-1) modified T7 probe, and Texas Red-BHQ-2-modified C7 probe.

Multiplex detection was performed by adding 10 or 5 μL of pretreated clinical sample directly into the multiplexed SLK reaction mix and then measured on the QuantStudio qPCR instrument for fluorescent readout at 56° C. (AscCas12b) or 60° C. (PAL5).

First derivative analysis was performed on the raw fluorescence values recorded every minute of the assay for each sample. The first derivative values indicate the slope of the fluorescence signal. The maximal slope value for the negative samples in an experiment was determined, and the SD of the slopes for the negative samples was calculated. The maximal slope value plus three SD values calculated from the negative samples was set as the cutoff value. The TTR (in minutes) was defined as the time at which the fluorescence of a sample surpassed the cutoff value in three consecutive recordings.

### Results

### RS9 Cas12a and AacCas12b

Activated RS9 (SLK9) cleaves both C7-probes and T7-probes enabling detection in both the FAM (green) and Texas Red (red) fluorescent channels. When AacCas12b is activated, it cleaves only T7 probes enabling detection in the FAM fluorescence channel only. Thus, both RS9 and AscCas 12b have the capacity to cleave T7-probes, whereas RS9 (SLK9) was found to only cleave C7 probes. Preferential probe cleavage enables determination of which Cas enzyme was activated (i.e., which target was present in the sample) (FIGS. 2A-B).

Positive samples show on signal in the Texas Red channel or on in both the FAM and Texas Red Channels, and negative samples show on signal in the FAM channel of off signal in the Texas Red channel. The assay is invalid if off signals are seen in both the FAM and Texas Red channels.

### RS9 Cas12a and PAL5 (SLK5-2)

RS9 (SLK9) was also paired with PAL5 (SLK5-2). Here, RNase Alert (InvitroGen) was used in the FAM (green) channel to indicate activated PAL5 (SLK5-2) enzyme detecting SARS-COV-2 and cleavage of C7-Texas Red in the red channel for the activation of RS9 (SLK9) and the detection of the internal control gene human RP. Within the PAL5 (SLK5-2)/RS9 (SLK9) system, a positive sample is indicated by an on signal in the green channel, regardless of the red channel status, whereas a negative sample shows on only in the red channel. In the absence of an on signal in either a green or red channel, the sample is invalid. Due to the greater thermostability of both enzymes, multiplex detection using PAL5 (SLK5-2)/RS9 (SLK9) was performed at 60° C. Both multiplex approaches were compared side-by-side with the present novel real-time SLK Direct approach using heat/ProteinaseK-treated ANS samples spiked with SARS-COV-2 ZeptoMetrix particles ranging from100 to 3.75cp/μL (FIGS. 3A-B).

In the RS9 (SLK9)/Aac system, SARS-COV-2 particles at 60cp/μL were detected in three of three replicates within 50 minutes using real-time SLK Direct. The PAL5 (SLK5-2)/RS9 (SLK9) real-time SLK Direct approach had a sensitivity of 5cp/μL and a TTR of under 30 minutes.

The real-time, multiplexed was validated using 41 extracted clinical samples, demonstrating 100% positive and 100% negative percent agreement with RT-qPCR results. Multiplexed real-time SLK Direct SLK Direct was validated with 10 NP/saline swab samples, demonstrating 100% positive and 100% negative percent agreement with RT-qPCR results.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A composition comprising a
(a) a first Cas enzyme having collateral cleavage activity and a first detectably labeled nucleic acid probe comprising a first reporter dye; and
(b) a second Cas enzyme having collateral cleavage activity and a second detectably labeled nucleic acid probe comprising a second reporter dye, wherein:
i. the first and second Cas enzyme are different; and
ii. the first and second detectably labeled nucleic acid probe are different,
wherein both the first and second Cas enzyme demonstrate preferential cleavage specificity for its corresponding first or second detectably labeled nucleic acid probe compared to its non-corresponding detectably labeled nucleic acid probe.

2. The composition of claim 1, wherein the first and second reporter dye are different.

3. The composition of claim 1, wherein the first and second reporter dye are the same.

4. The composition of any one of claims 1 to 3, wherein each detectably labeled nucleic acid probe is characterized by a sequence length of about 5 to about 15 nucleotides.

5. The composition of any one of claims 1 to 4, wherein the first Cas enzyme only cleaves the first detectably labeled nucleic acid probe and/or wherein the second Cas enzyme only cleaves the second detectably nucleic acid probe.

6. The composition of any one of claims 1 to 5, wherein at least one of the first or second Cas enzyme is a thermostable Cas enzyme.

7. The composition of any one of claims 1 to 6, wherein both the first and second Cas enzymes are thermostable Cas enzymes.

8. The composition of claim 6 or 7, wherein the thermostable Cas enzyme comprises the amino acid sequence of any one of SEQ ID NO: 1-11.

9. The composition of any one of claims 6 to 8, wherein the thermostable Cas enzyme comprises the amino acid sequence selected from the group consisting of SEQ ID NO: 5, SEQ ID NO: 9, SEQ ID NO: 10 and SEQ ID NO: 11.

10. The composition of any one of claims 5 to 9, wherein the first thermostable Cas enzyme comprises the amino acid sequence of SEQ ID NO: 11 and the second thermostable Cas enzyme comprises the amino acid sequence of any one of SEQ ID NO: 5, SEQ ID NO: 9, and SEQ ID NO: 10.

11. The composition of any one of claims 1 to 10, wherein the first and second detectably labeled nucleic acid probe have the same sequence length.

12. The composition of any one of claims 1 to 10, wherein the first and second detectably labeled nucleic acid probe have a different sequence length.

13. The composition of any one of claims 1 to 10, wherein the detectably labeled nucleic acid probe sequence lengths comprise about 7 to about 12 nucleotides.

14. The composition of claim 1, wherein at least one detectably labeled nucleic acid probe sequence is 7 nucleotides in length.

15. The composition of claim 1, wherein at least one detectably labeled nucleic acid probe sequence is 12 nucleotides in length.

16. The composition of claim 1, wherein the detectably labeled nucleic acid probe sequence is greater than 7 nucleotides and less than 12 nucleotides.

17. The composition of any one of claims 1 to 16, wherein the reporter dye is selected from the group consisting of: Cy5, FAM, TxRed, YakYel, and HEX.

18. The composition of any one of claims 1 to 17, wherein at least one reporter dye is or comprises Cy5.

19. The composition of any one of claims 1 to 17, wherein at least one reporter dye is or comprises FAM.

20. The composition of any one of claims 1 to 17, wherein at least one reporter dye is or comprises TxRed.

21. The composition of any one of claims 1 to 17, wherein at least one reporter dye is or comprises YakYel.

22. The composition of any one of claims 1 to 17, wherein at least one reporter dye is or comprises HEX.

23. The composition of any one of claims 1 to 17, wherein the detectably labeled nucleic acid probe sequence lengths are selected from the group consisting of 7 nucleotides and 12 nucleotides; and wherein the reporter dye is selected from the group consisting of: Cy5, FAM, TxRed, YakYel, and HEX.

24. The composition of any one of claims 1 to 23, wherein the first and second detectably labeled nucleic acid probe comprises the same nucleotides.

25. The composition of any one of claims 1 to 23, wherein the first and second detectably labeled nucleic acid probe comprises at least one nucleotide that differ.

26. The composition of any one of claims 1 to 23, wherein the first and second detectably labeled nucleic acid probe comprises different nucleotides.

27. The composition of any one of claims 1 to 26, wherein at least one detectably labeled nucleic acid probe sequence comprises at least one thymine nucleotide.

28. The composition of claim 27, wherein both detectably labeled nucleic acid probe sequences comprise at least one thymine nucleotide.

29. The composition of any one of claims 1 to 27, wherein at least one detectably labeled nucleic acid probe sequence comprises about 7 to about 12 thymine nucleotides.

30. The composition of claim 29, wherein both detectably labeled nucleic acid probe sequences comprise about 7 to about 12 thymine nucleotides.

31. The composition of any one of claims 1 to 26, wherein at least one detectably labeled nucleic acid probe sequence comprises of 95%-100% thymine nucleotides.

32. The composition of claim 31, wherein both detectably labeled nucleic acid probe sequences comprise of 95%-100% thymine nucleotides.

33. The composition of any one of claims 1 to 26, wherein at least one detectably labeled nucleic acid probe sequence comprises at least one cytosine nucleotide.

34. The composition of claim 33, wherein both detectably labeled nucleic acid probe sequences comprise at least one cytosine nucleotide.

35. The composition of any one of claims 1 to 26, wherein at least one detectably labeled nucleic acid probe sequence comprises about 7 to about 12 cytosine nucleotides.

36. The composition of claim 35, wherein both detectably labeled nucleic acid probe sequences comprise about 7 to about 12 cytosine nucleotides.

37. The composition of any one of claims 1 to 26, wherein at least one detectably labeled nucleic acid probe sequence comprises of 95%-100% cytosine nucleotides.

38. The composition of claim 37, wherein both detectably labeled nucleic acid probe sequences comprise of 95%-100% cytosine nucleotides.

39. The composition of claim 1, wherein the detectably labeled nucleic acid probes comprise a detectably labeled nucleic acid probe sequence and reporter dye selected from the group consisting of T7-Cyc5, C7-Cyc5, T7-TxRed, C7-TxRed, DNaseAlert-HEX, RNaseAlert-FAM and rN7-FAM.

40. The composition of any one of claims 1 to 39, wherein the detectably labeled nucleic acid probe further comprises a quencher.

41. The composition of claim 40, wherein the quencher is selected from the group consisting of 3IAbRQsp, BHQ2, 3IABKFQ, and BHQ1.

42. The composition of any one of claims 1 to 41, wherein composition further comprise a guide polynucleotide.

43. The composition of claim 42, wherein the guide polynucleotide comprises a polynucleotide sequence complementary to a target nucleic acid.

44. A method of detecting a target nucleic acid in a sample comprising:
a. contacting the sample with a composition comprising:
(a) a first Cas enzyme having collateral cleavage activity and a first detectably labeled nucleic acid probe comprising a first reporter dye;
(b) a second Cas enzyme having collateral cleavage activity and a second detectably labeled nucleic acid probe comprising a second reporter dye, wherein
i. the first and second detectably labeled nucleic acid probe are different; and
ii. the first and second Cas enzyme are different and wherein both the first and the second Cas enzyme demonstrates preferential cleavage specificity for its corresponding detectably labeled nucleic acid probe comparable to its non-corresponding detectably labeled nucleic acid probe, and
(c) a first and a second guide polynucleotide;
wherein hybridization of the first guide polynucleotide and first Cas enzyme or second guide polynucleotide and second Cas enzyme with a target nucleic acid activates the collateral activity and cleavage of a corresponding detectably labeled nucleic acid probe
wherein cleavage of a corresponding detectably labeled nucleic acid probe indicates presence of the target nucleic acid.

45. The method of claim 44, wherein the first and second reporter dye are different.

46. The method of claim 44, wherein the first and second reporter dye are the same.

47. The method of any one of claims 44 to 46, wherein each detectably labeled nucleic acid probe is characterized by a sequence length of about 5 to about 15 nucleotides

48. The method of any one of claims 44 to 47, wherein the method further comprises a step of incubating the sample with the composition.

49. The method of claim 48, wherein the step of incubating is performed for a period of time within a range of 10 seconds to 90 minutes.

50. The method of any one of claims 44 to 49, wherein the method further comprises a step of determining cleavage of the first or second detectably labeled nucleic acid probe.

51. The method of claim 50, wherein determining cleavage of the first or second detectably labeled nucleic acid probes comprises detecting a reporter dye signal.

52. The method of claim 51, wherein the reporter dye signal is a fluorescence signal.

53. The method of any one of claims 44 to 52, wherein at least one of the first or second Cas enzyme is a thermostable Cas enzyme.

54. The method of claim 53, wherein the first and second Cas enzymes are thermostable Cas enzymes.

55. The method of claim 53 or claim 54, wherein the thermostable Cas enzyme comprise the amino acid sequence of any one of SEQ ID NO: 1-11.

56. The method of claim 53, wherein the thermostable Cas enzymes comprise the amino acid sequence selected from the group consisting of SEQ ID NO: 5, SEQ ID NO: 9, SEQ ID NO: 10 and SEQ ID NO: 11.

57. The method of claim 54, wherein the first thermostable Cas enzyme comprises the amino acid sequence of SEQ ID NO: 11 and the second thermostable Cas enzyme comprises the amino acid sequence of any one of SEQ ID NO: 5, SEQ ID NO: 9, and SEQ ID NO: 10.

58. The method of any one of claims 44 to 57, wherein the first and second detectably labeled nucleic acid probe have the same sequence length.

59. The method of any one of claims 44 to 57, wherein the first and second detectably labeled nucleic acid probe have a different sequence length.

60. The method of any one of claims 44 to 59, wherein the detectably labeled nucleic acid probe sequence lengths comprise 7 to 12 nucleotides.

61. The method of claim 44, wherein at least one detectably labeled nucleic acid probe sequence is 7 nucleotides in length.

62. The method of claim 44, wherein at least one detectably labeled nucleic acid probe sequence is 12 nucleotides in length.

63. The method of claim 44, wherein the detectably labeled nucleic acid probe sequence is greater than 7 nucleotides and less than 12 nucleotides.

64. The method of any one of claims 44 to 63, wherein the reporter dye is selected from the group consisting of: Cy5, FAM, TxRed, YakYel, and HEX.

65. The method of any one of claims 44 to 64, wherein at least one reporter dye is or comprises Cy5.

66. The method of any one of claims 44 to 64, wherein at least one reporter dye is or comprises FAM.

67. The method of any one of claims 44 to 64, wherein at least one reporter dye is or comprises TxRed.

68. The method of any one of claims 44 to 64, wherein at least one reporter dye is or comprises YakYel.

69. The method of any one of claims 44 to 64, wherein at least one reporter dye is or comprises HEX.

70. The method of any one of claims 44 to 64, wherein the detectably labeled nucleic acid probe sequence lengths are selected from the group consisting of 7 nucleotides and 12 nucleotides and wherein the reporter dye is selected from the group consisting of: Cy5, FAM, TxRed, YakYel, and HEX.

71. The method of any one of claims 44 to 70, wherein at least one detectably labeled nucleic acid probe sequence comprises at least one thymine nucleotide.

72. The method of claim 71, wherein both detectably labeled nucleic acid probe sequences comprise at least one thymine nucleotide.

73. The method of any one of claims 44 to 71, wherein at least one detectably labeled nucleic acid probe sequence comprises about 7 to about 12 thymine nucleotides.

74. The method of claim 73, wherein both detectably labeled nucleic acid probe sequences comprise about 7 to about 12 thymine nucleotides.

75. The method of any one of claims 44 to 71, wherein at least one detectably labeled nucleic acid probe sequence comprises of 95%-100% thymine nucleotides.

76. The method of claim 75, wherein both detectably labeled nucleic acid probe sequences comprise of 95%-100% thymine nucleotides.

77. The method of any one of claims 44 to 70, wherein at least one detectably labeled nucleic acid probe sequence comprises at least one cytosine nucleotide.

78. The method of claim 77, wherein both detectably labeled nucleic acid probe sequences comprise at least one cytosine nucleotide.

79. The method of any one of claims 44 to 70, wherein at least one detectably labeled nucleic acid probe sequence comprises about 7 to about 12 cytosine nucleotides.

80. The method of claim 79, wherein both detectably labeled nucleic acid probe sequences comprise at in the range of about 7 to about 12 cytosine nucleotides.

81. The method of any one of claims 44 to 70, wherein at least one detectably labeled nucleic acid probe sequence comprises of 95%-100% cytosine nucleotides.

82. The method of claim 81, wherein both detectably labeled nucleic acid probe sequences comprise of 95%-100% cytosine nucleotides.

83. The method of claim 44, wherein the detectably labeled nucleic acid probes comprise a detectably labeled nucleic acid probe sequence and reporter dye selected from the group consisting of T7-Cyc5, C7-Cyc5, T7-TxRed, C7-TxRed, DNaseAlert-HEX, RNaseAlert-FAM and rN7-FAM.

84. The method of any one of claims 44 to 83, wherein at least one detectably labeled nucleic acid probe further comprises a quencher.

85. The method of claim 84, wherein both detectably labeled nucleic acid probe further comprise a quencher.

86. The method of claim 84 or 85, wherein the quencher is selected from the group consisting of 3IAbRQsp, BHQ2, 3IABKFQ, and BHQ1.

87. The method of any one of claims 44 to 86, wherein the sample comprises a cell-free extract.

88. The method of any one of claims 44 to 87, wherein the sample comprises a biological sample.

89. The method of claim 88, wherein the biological sample comprises a mammalian sample.

90. The method of claim 89, wherein the mammalian sample comprises a human sample.

91. The method of claim 88, wherein the biological sample comprises bone marrow, blood, blood cells, ascites, tissue, free floating nucleic acids, sputum, salvia, or urine.

92. The method of claim 88, wherein the biological sample comprises a viral sample.

93. The method of claim 88, wherein the biological sample comprises a microbial sample.

94. The method of any one of claims 44 to 93, wherein the sample comprises at least one target nucleic acid.

95. The method of claim 94, wherein the target nucleic acid comprises a DNA polynucleotide sequence.

96. The method of claim 94, wherein the target nucleic acid comprises an RNA polynucleotide sequence.

97. The method of any one of claims 44 to 96, wherein the guide polynucleotide comprises a polynucleotide sequence complementary to a target nucleic acid.

98. The method of any one of claims 44 to 97, wherein the first and second guide polynucleotide sequence are different.

99. The method of any one of claims 94 to 98, wherein upon contacting the first Cas enzyme and first guide polynucleotide or second Cas enzyme and second guide polynucleotide with the target nucleic acid, collateral cleavage activity is increased relative to an appropriate reference standard.

100. The method of claim 99, wherein the appropriate reference standard comprises a sample that does not comprise a target nucleic acid.

101. The method of claim 99, wherein the appropriate reference standard comprises a thermostable Cas enzyme with collateral activity further comprising a mutation that abolishes the collateral cleavage activity.

102. A kit comprising the composition of any one of claims 1 to 43.

103. The kit according to claim 102, further comprising a first guide polynucleotide and a second guide polynucleotide.
